(19) United States
(12) Patent Application Publication
(10) Pub. No.: US 2024/0409917 A1
(43) Pub. Date: Dec. 12, 2024

(54) THE MIDBODY AND MIDBODY REMNANT ARE ASSEMBLY SITES FOR RNA AND ACTIVE TRANSLATION

(71) Applicant: Wisconsin Alumni Research Foundation, Madison, WI (US)

(72) Inventors: Ahna Skop, Sun Prairie, WI (US); Sungjin Park, Madison, WI (US); Smit Patel, Madison, WI (US)

(21) Appl. No.: 18/741,343

(22) Filed: Jun. 12, 2024

Related U.S. Application Data
(60) Provisional application No. 63/507,671, filed on Jun. 12, 2023.

(51) Int. Cl. C12N 15/10; A61K 47/50; C12N 5/07; G01N 33/574

(52) U.S. Cl. CPC C12N 15/1003; A61K 47/50; C12N 5/06; G01N 33/574; C12N 2506/45

(57) ABSTRACT

Methods of isolating midbodies or midbody remnants by combining a polyethylene glycol (PEG) solution with a biological sample, incubating the PEG solution and the biological sample and recovering the midbodies or midbody remnants via low-speed centrifugation. The biological sample may be conditioned media, plasma, serum, cerebral spinal fluid, urine, blood, saliva or tissue. Methods of using the midbodies or midbody remnants are also provided.

### CROSS-REFERENCE TO RELATED APPLICATIONS

The present application claims priority to U.S. Provisional Patent Application No. 63/507,671, filed Jun. 12, 2023, the entire contents of which are hereby incorporated by reference.

### STATEMENT REGARDING FEDERALLY SPONSORED RESEARCH

This invention was made with government support under GM139695 awarded by the National Institutes of Health and under 1716298 awarded by the National Science Foundation. The government has certain rights in the invention.

### BACKGROUND OF THE INVENTION

The midbody (MB) is a protein and RNA-rich structure assembled during mitosis at the overlapping plus ends of spindle microtubules, where it recruits and positions the abscission machinery that separates dividing cells. Long thought to be quickly internally degraded in daughter cell endo-lysosomes, recent studies revealed that a majority of MBs are released extracellularly as membrane-bound particles, or large extracellular vesicles, following bilateral abscission from nascent daughter cells. Released post-mitotic MB remnants (MBRs) are bound and tethered by neighboring cells, internalized, and can persist in endosomal compartments for up to 48 hours as signaling organelles (termed MB-containing endosomes or MBsomes) before being degraded by endo-lysosomes.

Distinct cell types, including cancer and stem cells, exhibit differing avidities for internalizing MBRs, and exogenous addition of MBRs correlates with increased proliferation and tumorigenic behavior. MBRs have been implicated in specifying apicobasal polarity and lumenogenesis in epithelia; specifying primary cilium formation, neurite formation, and dorsoventral axis formation; and specifying stem cell pluripotency.

The functional importance of MBR signaling in the regulation of cell behavior, cell proliferation, architecture, and cell fate is only beginning to be understood. In view of the foregoing, it would be desirable to have a better understanding of MBR biogenesis, function and improved methods of isolation.

### BRIEF SUMMARY OF THE INVENTION

As demonstrate herein, large EVs originate during mitosis, are midbody remnants, and that MKLP1 and translation activity are unique markers for this EV class. Described here are methods of isolating midbodies, midbody remnants or large extracellular vesicles. In one embodiments the method comprises combining a polyethylene glycol (PEG) solution with a biological sample comprising midbodies or midbody remnants, incubating the PEG solution and the biological sample for at least 4 hours and recovering the midbodies or midbody remnants, wherein the PEG solution comprises between 0.5 and 5% PEG. In some embodiments, the PEG solution has a final concentration of at least 1% and less than 3%. In some embodiments, the PEG solution further comprises nanoparticles. In some embodiments the method further comprises labeling the recovered midbodies, midbody remnants or lEV with an MKLP1, CD9, MgcRACGAP1, PLK1, AURK, CITK, ANNEXIN 11, TEX14 or ARC affinity reagent and sorting for the labeled recovered midbodies or midbody remnants.

Another embodiment of the present invention provides a method for inducing the differentiation of cells. In some embodiments, the method comprises contacting a pluripotent cell with the midbodies or midbody remnants produced by a method described herein, wherein the pluripotent cell differentiates into the same cell type from which the midbodies or midbody remnants were derived.

Another embodiment of the present invention provides a method detecting cancer in a subject. In some embodiments, the method comprises obtaining a biological sample from a subject, isolating the midbodies or midbody remnants from the biological sample using a method described herein and counting the midbodies or midbody remnants to determine if the subject has cancer. In some embodiments, the presence of a biomarker in the isolated midbodies, MBR or lEV is indicative of cancer.

Another embodiment of the present invention provides a method of delivering a therapeutic cargo to a target cell. In some embodiments, the method comprises collecting midbodies, MBR or lEV by a method described herein and contacting the target cell with the collected midbodies, MBR or lEV.

Another embodiment of the present invention provides a method of diagnosing a proliferative disease. In some embodiments, the method comprises measuring MKLP1 in a biological sample from a subject and comparing the level of MKLP1 in the biological sample to a level of MKLP1 in a control sample, wherein an increase in the level of MKLP1 in the biological sample as compared to that in the control sample is indicative of the proliferative disease in the subject.

Another aspect of the present invention provides a method of selecting large extracellular vesicles (iEV) from a sample. In some embodiments, the method comprises contacting a sample with an anti-MKLP1 antibody, wherein the portion of the sample that binds to MKLP1 contains the lEV or MBR. In some embodiments, selecting the lEV or MBR isolates the lEV or MBR from the sample. In some embodiments, the portion of the sample that does not bind to the anti-MKLP1 antibody contains a small EV.

### BRIEF DESCRIPTION OF THE DRAWINGS

The patent or application file contains at least one drawing executed in color. Copies of this patent or patent application with color drawing(s) will be provided by the Office upon request and payment of the necessary fec.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Increasing the concentration of PEG6000 distorts the MKLP1-GFP signal and MBR structure. (A) Cleared conditioned medium from HeLa (CCL2) cells was incubated with increasing concentrations of PEG6000 and settled onto coverslips. Increasing PEG concentration resulted in decreased transparency. The Scale bar represents 5 um. (B) Higher PEG6000 concentrations caused an increased, blurred appearance to the large vesicles. The coverslips were imaged at 20× magnification on a brightfield microscope. The scale bar represents 100 μm. (C) MBRs isolated from HeLa-MKLP1-GFP cell culture medium by increasing PEG6000 concentrations imaged at 100× magnification by N-SIM Z projection. The scale bar represents 10 μm.

FIG. 2. Isolated MBRs are midbodies (MB) formed during the late telophase stage of mitosis. (A) Representative images of MBRs isolated from HeLa-CCL-2 cells stained with anti-MKLP1 (magenta) and anti-CD9 (cyan). Images were taken at 60× magnification on a confocal microscope. The scale bar represents 24 μm, and the inset represents an 8× enlargement of the dotted square. The same slide images of isolated MBRs from HeLa-CCL-2 cell culture labeled with anti-MKLP1 (Magenta) and anti-CD9 (Cyan) were taken at 100× magnification on an N-SIM microscope. The scale bar represents 10 μm, and the inset represents a 4× enlargement of the dotted square. (B) Representative images of mitotic HeLa-CCL-2 cells labeled with anti-MKLP1 (green), anti-α-tubulin 555 (red), and DAPI (gray). The green midbody in the first image attaches two daughter cells in the final stages of mitosis. Green MBR is taken up by a cell in interphase post-mitosis. Images were taken at 100× magnification on an N-SIM microscope. The scale bar represents 10 μm. (C) Representative images of isolated MBRs from HeLa-CCL-2 cells using transmission electron microscopy confirm the electron-dense property of MBs at 40,000× magnification. The scale bar represents 500 nm.

FIG. 3. Comparison of GOLD PEG5000 nanoparticle concentrations in the isolation of midbody remnants (MBRs). (A) HeLa CCL-2 clear conditioned medium was incubated with decreasing concentrations of PEG-coated gold nanoparticles overnight at 4° C. MBRs were stained with anti-MKLP1 and imaged at 20× magnification. The scale bar represents 90 μm. (B) HeLa MKLP1-GFP clear conditioned medium was incubated with PEG5000-coated gold nanoparticles at a 1:5000 (v/v) dilution overnight at 4° C. MBRs were stained with anti-CD9 and imaged at 20× magnification. The scale bar represents 90 μm. (C) HeLa MKLP1-GFP clear conditioned medium was incubated with PEGylated gold nanoparticles at a 1:5000 dilution (v/v) overnight at 4° C. MBRs were stained with anti-CD9 and imaged at 100× magnification. The scale bar represents 10 μm. Inset shows a 10× zoomed view of the MBR.

FIG. 4. Pictorial overview of a protocol for the isolation of class III extracellular vesicles or midbody remnants from human cell culture medium using 1.5% PEG6000 or PEGylated gold particles.

FIG. 5: Canonical Extracellular vesicle (EV) markers CD9, CD81, flotillin-1, and dsRNA assemble on to the spindle midzone, midbody, and MBR during mitosis. (A-D) EV and midbody marker (MKLP1) localization patterns during each stage of mitosis. HeLa (CCL2) cells were immunostained with EV markers, shown in cyan, including (A) CD9, (B) CD81, (C) Flotillin-1, and (D) dsRNA. MKLP1 is shown in magenta, and DAPI is shown in grayscale in all panels. (A-C) CD9, CD8 and flotillin form a clouds on either side of the metaphase plate, and then assemble on to the spindle midzone, midbody, and then MBR. (D) dsRNA does not appear as a cloud but in puncta in the cytoplasm during interphase and metaphase. Then in anaphase, dsRNA localizes to the spindle midzone, midbody and MBR. Overall, the canonical markers used by the EV field assemble on the spindle midzone, midbody and MBR.

Images were taken with a 100× objective on a SIM microscope. Insets show a 15× zoom of the dashed box in the image to reveal co-localization of the EV marker (cyan) with the midbody marker, MKLP1 (magenta). The percentages indicate the fraction of cells with the same protein localization pattern for each EV marker. For the following stages and markers, N represents the number of images analyzed. CD9: interphase, n=11; metaphase, n=18; anaphase, n=15, early telophase, n=15; late telophase, n=14; MBR, n=15. CD81: interphase, n=13; metaphase, n=13; anaphase, n=13; early telophase, n=13; late telophase, n=14; MBR, n=13. Flotillin-1: interphase, n=14; metaphase, n=16; anaphase, n=8; early telophase, n=13; late telophase, n=15; MBR, n=13. dsRNA: interphase, n=13; metaphase, n=13; anaphase, n=13; early telophase, n=13; late telophase, n=13; MBR, n=13. The scale bar represents 10μm.

FIG. 6: Localization of the canonical EV markers CD9, CD63 and CD81 with MKLP1 in isolated MBRs. Isolated HeLa MBRs (using 1.5% PEG6000 method) were immunostained with EV markers (A) CD9, (B) CD63, and (C) CD81 labeled in cyan; and MKLP1 labeled in magenta. Here, we observe that for CD9 (A), this marker is found primarily within the MBR, whereas, CD63 and CD81 (B-C), were found primarily on the surface of the MBR. (D-F) Normalized line scans of MBRs in projected Z-series images from (A-C). n represents the number of MBRs analyzed. Images were taken with 100 Å˜ objective on a SIM microscope. Z-stack images were taken at 0.15 μm intervals through the whole MBR. The scale bar represents 1 um.

FIG. 7: Schematic summary of common and newly developed EV isolation methods used in this study. EVs/MBRs were isolated from cell culture media using ultracentrifugation, total exosome isolation reagent (TEIR), Protein G Dynabeads (Dynabeads), Skop Laboratory 1.5% PEG6000 (Skop 1.5% PEG), and Skop Laboratory 30 nm gold particle methods (Skop gold nanoparticles). Isolated EVs/MBRs were then spotted on Poly-L-Lysine coverslips, immunostained and then imaged on a SIM, confocal, or brightfield microscope.

FIG. 8: Common, commercial, and newly developed isolation methods all isolate midbody remnants (MBRs). (A) Using common, commercial, and newly developed methods for isolating EVs, we determined if CD9 (cyan) and MKLP1 (magenta). were detected in isolated fixed samples. Here, all isolations demonstrated a considerable overlap between MKLP1 and CD9 labeling, but not all CD9 positive EVs were MKLP1 positive. Insets show a 5× zoom of the dashed box in the image. Images were taken with a 60× objective, and the scale bar represents 50 μm. The number of individual and overlapping particles in each channel were automatically counted using a custom macro tool using ImageJ developed in the lab (see Particle counting in methods) and data can be found in (B-C). (B) Percentage of MKLP1 labeled particles (MBRs) that were also labeled with CD9 by each method were quantified. Data show clearly that all EV isolation methods isolate MBRs. (C) In general, the percentage of MKLP1 labeled particles (MBRs) among all isolated large particles (CD9, MKLP1, and overlapping) for each method is just over 45%. For our Skop lab gold nanoparticle method, this increased to about 65%. For the following, N represents the number of images analyzed: ultracentrifuge (n=15), TEIR (n=19), Dynabeads (n=15), Skop 1.5% PEG6000 (n=33), and Skop gold nanoparticles (n=17). Asterisk represents statistical significance (*p<0.05, **p<0.01). Numbers on graph bars represents the exact percentage on the Y axis. Dotted red line represents the control ultracentrifuge percentage.

FIG. 9: Cytokinetic failure and mitotic inhibition reduce total EV production including MBRs. (A) Schematic representation of MBR isolation and cell fixation following treatment with siRNA (48 hours) or G2/M chemical blocker RO-3306 (24 hour). (B) EV classification for quantitative analysis. CD63 positive (magenta) and MKLP1 positive (green) vesicles are classified as MBRs (>0.8 um). MKLP1 negative, CD63 positive EVs fall into three classes: IEV (large EVs, >0.8 um), mEV (medium EVs, 0.5-0.8 um) and sEV (small EVs, <0.5 um). Scale Bar represent 1 μm. (C) HeLa cells were treated with Control siRNA or MKLP1 siRNA and labeled with anti-MKLP1 (green), phalloidin (red), anti-α tubulin (magenta), and DAPI (cyan). Scale bars represent 100 μm. (D-F) Quantification of total isolated EVs in MKLP1, CD9, and overlapping channels were counted per unit area (440 μm×360 μm). Knockdown of MKLP1 led to a significant decrease in all sizes of EVs, not just MBRs. For the following, N represents the number of images analyzed: Control siRNA (n=19), and MKLP1 siRNA (n=30). (G) Hela cells were treated with DMSO (solvent control) or RO-3306 and labeled with anti-MKLP1 (green), phalloidin (red), anti-α tubulin (magenta), and DAPI (cyan). Scale bars represent 100 μm. (H-J) Quantification of total isolated EVs in MKLP1, CD63, and overlapping channels were counted per unit area (440 μm×360 μm). RO-3306 treatment led to a significant decrease in all sizes of EVs, not just MBRs. For the following, N represents the number of images analyzed: DMSO control (n=30), and RO-3306 (n=30). Statistical analysis was conducted using an unpaired t-test to compare the differences in total isolated EV production between the treatment groups (*p<0.05, **p<0.01, ***p<0.001, ****p<0.0001). All images were taken with a 20× objective with an NA of 0.8 on an ECHO Revolve brightfield microscope.

FIG. 10: Common and newly developed methods all isolate and preserve translating MBRs. (A) Schematic showing anti-puromycin labeling strategy on isolated MBRs. (B) Isolated MBRs were treated with puromycin and labeled with anti-puromycin (cyan) and MKLP1 (magenta). All isolations demonstrated overlap between puromycin and MKLP1 labeling, confirming that all methods are isolating and preserving translating MBRs. Particles in MKLP1, puromycin, and overlapping channels were counted. Merged image represents a 3.5× zoom of dashed box in CD9 (cyan) and MKLP1 (m) images. Dashed inset represents a 40× zoomed image of the small-dashed box in merged image. Dotted inset in upper left corner of MERGE with CYAN only reveals EVs that only label with PURO. Images were taken with a 60× objective, scale bars represent 50 μm. (C) Percentage of MKLP1 labeled particles (MBRs) labeled with puromycin before isolation. For the following, N represents the number of images analyzed: ultracentrifuge (n=15), TEIR (n=14), Dynabeads (n=15), Skop Laboratory 1.5% PEG6000 (n=25), and Skop Laboratory 30 nm gold particles (n=26). (D) Percentage of MKLP1 labeled particles (MBRs) also labeled with puromycin after isolation. For the following, N represents the number of images analyzed: ultracentrifuge (n=15), TEIR (n=15), Dynabeads (n=15), Skop Laboratory 1.5% PEG6000 (n=15), and Skop Laboratory 30 nm gold particles (n=15). Numbers on graph bars represents the exact percentage on the Y axis. Dotted red represents the control ultracentrifuge percentage.

FIG. 11. The biogenesis of large translating EVs occurs in mitosis. Model depicting the assembly and localization patterns of the canonical EV markers, CD9, CD81, and flotillin-1, during mitosis (blue) and a summary of the class of EVs identified in our study. Here, CD9, CD81, and flotillin-1 localize as puncta in interphase cells, as two clouds around the metaphase plate and then assemble on the spindle midzone in anaphase cells. During anaphase, CD9, CD81, flotillin-1, and dsRNA each colocalize with MKLP1 (pink), the master protein necessary for cytokinesis and midbody function. During early and late telophase, CD9, CD81, flotillin-1, dsRNA, and MKLP1 are enriched in the midbody and newly forming MBR. During abscission, generation of the MBR occurs, and translation activity (shown by ribosomes) is observed. Top inset shows the generation of lEVs, mEVs, and sEV during interphase that we observed in FIG. 9. We reveal that blocks in mitosis or the cell cycle, lead to significant decreases in lEVs and MBRs and also mEVs, suggesting that their biogenesis primarily occurs in mitosis.

FIG. 12: Common EV markers colocalize with MKLP1 in late telophase and cytokinesis stages of mitosis. HeLa cells were labeled with proteins associated with EVs and cytokinesis including: CD81 (A), CD63 (B), HSP90 (C), ARF6 (D), TSG101 (E), ALIX (F), CIT-K (G), and PLK1 (H) are all labeled in red; MKLP1 is labeled in green; and tubulin is labeled in magenta. Images of the midbody in late telophase and MBR stage were taken, and localization of EV proteins relative to MKLP1 was observed. Images were taken with 100× objective on a SIM microscope. Scale bar represents 1 μm.

### DETAILED DESCRIPTION OF THE INVENTION

Extracellular vesicles (EVs) are particles that are released from almost every type of cell, are bound by a lipid bilayer, and function in cell-to-cell communication. EVs are currently classified into four distinct groups based on size: 1) exosomes or small EVs (40-100 nm), 2) microvesicles (100-1000 nm), 3) large EVs (1,000-2,000 nm), and 4) apoptotic bodies (1,000-10,000 nm). The biogenesis of exosomes is well understood, and apoptotic bodies are generated by dying cells. However, the biogenesis of microvesicles and large EVs are less understood. Despite seeking to standardize terminology, methods, and reporting across studies, most researchers have assumed that all EVs originate from interphase cells either by budding (for microvesicles or microparticles) or exocytosis (for exosomes and large EVs). However, the inventors surmised that cells are unlikely to generate a 1-2 μm large EVs during interphase due in part to the biophysical and physiological aspects of the biology of cells. In addition, the only time a cell normally generates a 1-2 μm vesicle is during mitosis at abscission, which produces the midbody remnant (MBR). As the inventors demonstrate herein, large EVs originate during mitosis, arc midbody remnants, and that MKLP1 and translation activity are unique markers for this EV class.

The midbody (MB) is a transient structure at the spindle midzone that is required for cytokinesis, the terminal stage of cell division. The midbody was long considered a vestigial remnant of cytokinesis but it is now known that MBs are released post-abscission as extracellular vesicles called MB Remnants (MBRs). MBR can be degraded by autophagy or be inherited by a newly-formed daughter cell, or nearby cell where they can accumulate intracellularly. Once engulfed by daughter cells or nearby cells, MBR can influence cell signaling and cell fate. Midbodies have also been linked to several neurological disorders, including microcephaly and cancers. Inefficient isolation techniques have prevented the discovery of biological mechanisms associated with large extracellular vesicles or MBRs. Disclosed herein are novel MB and MBR isolation methods and uses thereof.

MB remnants (MBRs) and can modulate cell proliferation, fate decisions, tissue polarity, neuronal architecture, and tumorigenic behavior. Here, the inventors demonstrate that the MB matrix—the structurally amorphous MB core of unknown composition—is the site of ribonucleoprotein assembly and is enriched in mRNAs that encode proteins involved in cell fate, oncogenesis, and pluripotency, that the inventors are calling the MB granule. Using a quantitative transcriptomic approach, the inventors identified a population of mRNAs enriched in mitotic MBs and confirmed their presence in signaling MBR vesicles released by abscission. The MB granule is unique in that it is translationally active, contains both small and large ribosomal subunits, and has both membrane-less and membrane-bound states. Both MBs and post-abscission MBRs are sites of spatiotemporally regulated translation, which is initiated when nascent daughter cells re-enter G1 and continues after extracellular release. The inventors demonstrate that the MB is the assembly site of an RNP granule. MKLP1 and ARC are necessary for the localization and translation of RNA in the MB dark zone, whereas ESCRT-III was necessary to maintain translation levels in the MB. Data presented herein suggest a model in which the MB functions as a novel RNA-based organelle with a uniquely complex life cycle. The inventors present a model in which the assembly and transfer of RNP complexes are central to post-mitotic MBR function and suggest the MBR serves as a novel mode of RNA-based intercellular communication with a defined biogenesis that is coupled to abscission, and inherently links cell division status with signaling capacity.

The midbody and midbody remnants are in a unique place to serve as not only a vehicle for delivering RNA based therapeutics, but an organelle where a therapeutic can also be synthesized or used for delivery to cells. This translation event occurs in early G1 of the next cell cycle just before abscission takes place. The ability to collect and engineer MBRs allows for this EV to be useful for EV-based cancer therapeutics and drug delivery. The inventors have also identified several genes including MKLP1, ARC (repurposed capsid-like RBP) that can be used to identify or isolate the MB or MBRs.

One aspect of the present disclosure provides a method of isolating midbodies (MB), midbody remnants (MBR) and large extracellular vesicles (lEV) comprising combining a polyethylene glycol (PEG) solution with a biological sample comprising MBs, MBRs or EV. The combination of the PEG solution and the biological sample is then incubated for at least 4 hours and as long as one week. The midbodies or midbody remnants are then recovered from the combination.

As used herein, a biological sample may be any biological fluid or tissue from which the isolation of MB or MBR is desired. In some embodiment, the biological sample may comprise conditioned media, plasma, serum, cerebral spinal fluid, urine, blood, saliva or tissue. Conditioned media is the media in which a cell or ex-vivo tissue is growing. Conditioned media comprises the secretome of a cell or tissue in culture, including all proteins and vesicles secreted into a culture media. Conditioned media can be collected from a primary cell or a cell line. The cell or cell line may be engineered such that it expresses a target of interest, for example a cell line could be engineered to express a therapeutic agent or cargo. In some embodiments, the conditioned media will comprise MB and or MBR which can be isolated with the methods described herein. To optimize isolation the conditioned medium can be isolated from actively growing cells (i.e., cells actively undergoing mitosis and thus producing MBs and MBRs). The conditioned media may be harvested by decanting media from a culture of actively growing adherent cells or may be collected by centrifuging cells and collecting the cell-free supernatant (conditioned media) from the cells. The centrifugation at this step is sufficient to remove cells and cell debris but need not be an ultra-centrifuge and can rely on a table-top centrifuge for example at 2000×g to 5000×g for 5-15 minutes. A biological sample may also comprise a biofluid, including, but not limited to blood, bile, bone marrow aspirate, breast milk, buffy coat, biopsy, cerebral spinal fluid (CSF), isolated cells, plasma, peripheral blood mononuclear cells, saliva, serum, sputum, stool, swabs (oral, nasal, vaginal fluids), cerebral spinal fluid, tissues, synovial fluid or urine. A biological sample may also comprise tissue, or any other sample comprising cells wherein the isolation of MB or MBR is desired. These biological fluids may also be prepared in the same means as cells in culture by collecting for example cell-free serum or by incubating cells such as white or red blood cells in media for 4 hours to 24 hours and then collecting the conditioned media from these cells as the biological fluid.

The biological fluid is then combined with a PEG solution. Combining denotes diluting the biological sample with a 2× to 5× concentration of the PEG solution to arrive at a final concentration of PEG in the combination of between 0.5% and 5% PEG (wt/v). As used herein, a polyethylene glycol (PEG) also known as polyethylene oxide (PEO) or poly (oxyethylene) (POE), is a synthetic, hydrophilic and biocompatible polyether. Typically, materials with molecular weight less than 20,000 g/mol are referred to as PEGs, whereas those with molecular weights above 20,000 g/mol are referred to as PEOs. The PEG may have a molecular weight in a range of about 400 g/mol to about 10,000 g/mol, or from 1,000 g/mol to 9000 g/mol, or from 2,000 g/mol to 8,000 g/mol, or from 4,000 g/mol to 7,000 g/mol. The average molecular weight may be between 5,000 g/mol to 7,000 g/mol, such as PEG6000. These polymers are soluble in water as well as in many organic solvents, such as ethanol, acetonitrile, toluene, acetone, dichloromethane, hexane, and chloroform. In the disclosed methods, PEG may aid in the precipitation of the MB or MBR. PEG may be in a solution, wherein the PEG may be at a final concentration of at least 0.5%, 1%, 1.5%, 2%, 2.5%, 3%, 4% or 5%. In preferred embodiments, the PEG may have a final concentration of 1.0%-3%, suitably 1.5%. In some embodiments, the PEG may have a molecular weight in a range of about 5000 g/mol to about 7000/mol.

In some embodiments, the PEG solution may further comprise nanoparticles. The nanoparticles may be metal nanoparticles. Metal nanoparticles may comprise silver, gold, palladium, titanium, zinc, or copper. The metal nanoparticles may have magnetic properties, such as superparamagnetic iron oxide nanoparticles (SPION). The metal nanoparticles may be of varying size for example from about 3 nm to about 50 nm in size. The nanoparticles found to work well in the Examples were 10 nm and 30 nm in size. The surface of the nanoparticles may be coated, modified or functionalized to optimize the function of the nanoparticle. For example, the nanoparticle may be coated with PEG of different molecular weights. In some embodiments, gold or iron oxide nanoparticles with PEG 5000 may be used. In some embodiments the gold particle concentration ranges from 0.01% to 0.1% (v/v), preferable 0.02% (v/v).

In some embodiments, the PEG solution and biological sample are combined and incubated together. The combination may be incubated for at least 4 hrs, 6 hrs, 8 hrs, 10 hrs, 12 hrs, 14 hrs, 16 hrs, 18 hrs, 20 hrs, 24 hrs or any amount of time in between. The inventors have demonstrated that the incubation can be maintained for up to one week. The combination may be incubated at 10° C., 8° C., 6° C., 4° C. or 2° C., or any temperature in-between. The incubation time and temperature may be adjusted accordingly, for example, a shorter incubation time may occur with a higher temperature. The inventors also demonstrate that the conditioned media may be frozen prior to incubation with PEG and the MB and MBRs can still be isolated at a later date.

In some embodiments the MB, MBR or EV are recovered from the combined PEG solution and biological sample by gravity collection on a slide or coverslip, via centrifugation or using a magnet if the iron oxide nanoparticles are included. The combination may be centrifuged at any speed in the range of about 5000×g, 4000×g, 3000×g, 2000×g, 1000×g, 500×g, 400×g or 300×g or any speed in-between. The combination may be centrifuged at the defined speed for any time in a range of about 20 min, 15 min, 10 min, or 5 min or any time in-between. The speed and time of the centrifugation may be adjusted accordingly for optimal recovery of MB, MBR or EV, for example, a slower centrifugation speed with increased time. Unlike prior methods, this method does not require the use of ultra-centrifugation at any point in the method. Thus, the centrifugation steps do not require speeds in excess of 50,000 rpm. For magnetic separation, magnetic separation techniques are well known in the art and can be used to collect the MBs, MBRs or EVs.

In some embodiments, the MB, MBR or EVs are labeled with a mitotic kinesin-like protein (MKLP1) affinity reagent. As disclosed herein, the inventors of the present disclosure have found MKLP1 is a specific marker for MB and distinguishes them from other extracellular vesicles including small EVs. The MKLP1 may be used to aid in the recovery of the large EVs, MB or MBR, for example with the use of cell sorting techniques, including fluorescent activated cell sorting, magnetic cell sorting or adhesion (affinity)-based cell sorting. In some embodiments, other markers of MB or MBR may be used, including, but not limited to ARC, ESCRT-III, CD9, CD63, CD81, HSP90, ALIX, TSG-101, RACGAP1, MgcRACGAP1, PLK1, AURK, CITK, ANNEXIN 11, TEX14, KLF4, FOS, JUN, ZFP36 or any other midbody antibodies known in the art. These markers may be used in isolation or combination with each other or additional markers such as CD9, CD63 and or CD81.

MB, MBR or EV isolated with the methods described herein may comprise any proteins or nucleic acids from the cell from which it was formed. As described herein, the MB, MBR or EV isolated by the present methods may comprise ribosomal subunits and RNA. The MB, MBR or EV associated ribosomal subunits, including both small and large subunits, are translationally active and are capable of translating the mRNA to generate protein.

In some embodiments, the method further comprises detecting the presence of RNA in the MB, MBR or EV. RNA may be detected by any means known in the art, including but not limited to Northern blot analysis, nuclease protection assay, in situ hybridization, antibodies, probes and or reverse transcription-polymerase chain reaction. The RNA may be derived from any source, including, but not limited to pathogenic RNA such as RNA derived from a virus with an RNA based genome such as RNA derived from Zika virus, West Nile virus, Chikungunya virus, Murine Leukemia Virus, or Human papillomavirus virus, or RNA associated with markers of cancer such as FOS/Jun or KLF4. The RNA may comprise any type of RNA, including, but not limited to coding RNA such as messenger RNA, non-coding RNA, such as ribosomal RNA, transfer RNA, small nuclear RNA, small nucleolar RNA, piwi-interacting RNA, microRNA or long noncoding RNA or circular RNA. The RNA may also comprise synthetic RNA such as guide RNA, CRISPR RNA, or tracer RNA. The RNA may also comprise an RNA expression profile, such that the expression, expression pattern or quantity of expression may be detected.

MB, MBR or EVs isolated by the methods provided herein may be used for intracellular signaling, for example in cell reprograming and tumorigenesis. Another aspect of the present disclosure provides a method for inducing the differentiation of cells comprising contacting a pluripotent cell with the midbodies or midbody remnants produced by the method described herein wherein the pluripotent cell differentiates into the same cell type from which the midbodies or midbody remnants were derived. Pluripotent cells have the capacity to divide and differentiate into other cell types. Contacting a pluripotent stem cell with a MB, MBR or EV isolated from a specified cell type may induce the differentiation of the pluripotent cell. In particular, the pluripotent stem cell may differentiate into a cell type that is the same as or a progenitor to, the cell type from which the MB, MBR or EV was isolated.

Another aspect of the present disclosure provides a method detecting cancer in a subject comprising obtaining a biological sample from a subject and isolating the midbodies or midbody remnants with the method described herein and counting the midbodies or midbody remnants to determine if the subject has cancer. MB, MBR or EV may be released from cancerous cells at a higher rate than from non-cancer cells because the cancer cells are actively dividing and thus isolating MB, MBRs or EVs from a set amount of serum or other biological or cellular sample from a subject may be used to measure the number or amount of MB or MBRs over a particular amount of time and may be used to monitor for an abnormally high amount of MB or MBRs as indicative of cancer. These MB or MBR may be released in greater amounts or frequency than noncancerous cells. MB or MBR released from cancer cells may comprise nucleic acids which comprise mutations commonly associated with cancer or transformed cells. MB or MBR released from cancer cells may accumulate in or be taken up by other cells. MB or MBR released from cancer cells may change the fate, identity or proliferative capacity of cells that take up the MB or MBR and thus could be a source of metastases. MBs derived from cancer cells may also comprise unique markers only found in certain cancers and thus may be used for diagnosis, prognosis or surveillance for recurrence. In some embodiments MKLP1 may be used to measure or count the MB or MBR.

Another aspect of the present disclosure provides a method of delivering a therapeutic cargo to a target cell comprising isolating midbodies or midbody remnants from a cell via the method described herein and contacting a target cell with the isolated midbodies or midbody remnants. In some embodiments, a cell may be modified to express a therapeutic cargo prior to isolating MB, MBR or EV from it. A cell may be modified by any means known in the art, including but not limited to transfection, electroporation, microinjection, transduction, transformation or diffusion or any means of genetic engineering including CRISPR/Cas based engineering of cells. In some embodiments the therapeutic cargo may comprise RNA, DNA, protein or small molecules. The MB, MBR or EV may be isolated with the methods described herein and the therapeutic cargo may be detected with the isolated MB or MBR. In some embodiments, contacting a target cell with the isolated MB, MBR or EV which comprise a therapeutic cargo will deliver the therapeutic cargo to the target cell. Cells tend to take up MB and MBR from cells of similar cell type so that may provide a means of targeting a therapeutic to a particular cell type by harvesting MB, MBR or EV from the same type of cell to which one wants to deliver a therapeutic.

Another aspect of the present disclosure provides a method of diagnosing a proliferative disease. In some embodiments, the method comprises measuring MKLP1 in a biological sample from a subject and comparing the level of MKLP1 in the biological sample to the level of MKLP1 in a control sample. In some embodiments, an increase in the level of MKLP1 in the biological sample as compared to that in the control is indicative of the proliferative disease in the subject. A proliferative disease is a disease or condition in which cells grow and divide resulting in an increased number of cells beyond what is expected and where the increased number of cells contributes to disease pathogenesis. Without limitation proliferative disease include, cancer, atherosclerosis, rheumatoid arthritis, psoriasis, idiopathic pulmonary fibrosis, scleroderma and cirrhosis of the liver. In some embodiments, the biological sample comprises plasma, serum, cerebral spinal fluid, urine, blood, salvia and tissue.

Another aspect of the present disclosure provides a method of selecting a MB, MBR or EV from a sample. In some embodiments, the method comprises contacting a sample with an anti-MKLP1 antibody, wherein the portion of the sample that binds to MKLP1 contains the large EV, midbody or MBR. In some embodiments selecting the large EV, MB or MBR isolates the large EV, MB or MBR from the sample. In some embodiments, the portion of the sample that does not bind to the anti-MKLP1 antibody contains a small EV. In some embodiments, the method further comprises collecting the small EVs. In some embodiments, the sample is selected from the group consisting of cell culture media, plasma, serum, cerebral spinal fluid, urine, blood, saliva and tissue.

### Additional Definitions

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following Examples are illustrative and should not be interpreted to limit the scope of the claimed subject matter.

### Example 1—A Protocol for the Isolation and Imaging of Class III Extracellular Vesicles or Midbody Remnants From Human Cell Culture Medium Using 1.5% PEG6000 and MKLP1 as a Marker

Reference is made to the manuscript: Park et al., “A protocol for isolating and imaging large extracellular vesicles or midbody remnants from mammalian cell culture,” the content of which is incorporated herein by reference in its entirety.

Traditionally, midbody remnants (MBRs) are isolated from cell culture medium using ultracentrifugation, which is expensive and time consuming. Here, we pre-sent a protocol for isolating MBRs or large extracellular vesicles (EVs) from mammalian cell culture using either 1.5% polyethylene glycol 6000 (PEG6000) or PEG5000-coated gold nanoparticles. We describe steps for growing cells, collecting media, and precipitating MBRs and EVs from cell culture medium. We then detail characterization of MBRs through immunofluorescent antibody staining and immunofluorescent imaging.

### Before You Begin

The protocol below describes the specific steps for isolating midbody remnants (MBRs), or large extracellular vesicles (EVs), from the culture medium of human cells using polyethylene glycol 6000 (PEG6000), or PEG5000 coated gold nanoparticles, precipitation. This protocol is optimized for HeLa-CCL-2 cells, HeLa Kyoto-mitotic kinesin-like protein 1 (MKLP1)-GFP cells¹ and Dulbecco's Modified Eagle Medium (DMEM)/F12 culture medium. It has also been successfully used with DMEM culture medium. Other cell lines and culture media may be suitable alternatives; however, additional protocol modifications may be necessary. All cell procedures are performed in a Class II biosafety cabinet using sterile technique. The HeLa cells are thawed at least 7 days before MBR isolation and cultured in a 37° C. humidified incubator with 5% CO₂, with regular passaging. The cells are regularly screened for mycoplasma.

Before beginning the isolation, prepare medium, solutions, and poly-L-lysine coated coverslips.

### Preparation of Poly-L-Lysine Coating of Cover Glasses (Coverslips)

### Timing: 1 Day

MBRs, or large EVs, are small particles that do not adhere well to glass. Thorough cleaning of the glass coverslips with sterile water and sonication removes any surface contamination. Treating the coverslip with poly-L-lysine promotes adhesion of MBRs by electrostatic interactions between the cell membrane and the poly-L-lysine. Attachment to the coverslip is essential for the later stages of the protocol which involve immunostaining of MBRs (see problem 1).
1. Wash 18-mm circular coverslips in sterile distilled water.
2. Sonicate the coverslip in 1 M KOH solution for 30 min.
3. Wash five times with sterile distilled water.
4. Wash once with 100% ethanol and dry.
5. Place each coverslip into a well of a 12-well plate.
6. Cover the surface of each coverslip with 50 mL poly-L-lysine and incubate at either 2° C.-8° C. for 12-18 h or 37° C. for 1 h.
7. Wash the coverslips three times with sterile distilled water.
8. Store the coated coverslips at-200 C for up to 6 months.

Note: Perform steps 3-8 in a Biosafety cabinet using sterile technique and forceps to handle the coverslips. Wash the coverslips by dipping into the relevant solution.

### Preparation of Reynolds Lead Citrate Solution for Electron Microscopy

### Timing: 1 h

Reynolds lead citrate is used as an enhancer for heavy metal staining in electron microscopy. One of the techniques used to identify isolated MBRs is imaging with a transition electron microscope.
9. Add 1.33 g of lead nitrate (80 mM) and 1.76 g of sodium citrate dihydrate (120 mM) to 30 mL of sterile distilled water.
10. Shake vigorously and incubate for 30 min at 20° C.-25° C. with intermittent mixing.
11. Add 8 mL of 1 N sodium hydroxide and bring up to a final volume of 50 mL with sterile distilled water.
12. Store the solution in a glass bottle at 20° C.-25° C. for up to 6 months.

TABLE 2
Key Resources Table

| | | |
|---|---|---|
| REAGENT or RESOURCE | SOURCE | IDENTIFIER |

Antibodies

| | | |
|---|---|---|
| Rabbit Anti-MKLP1 | Novus Biologicals | Cat#NBP2-56923 |
| Mouse Anti-CD9 | Santa Cruz | Cat#SC-13118 |
| | Biotechnology | |
| Mouse Anti-a-tubulin Alexa Fluor 555 | Millipore Sigma | Cat#05-829X-555 |
| Alexa Fluor 488 Goat anti-Rabbit IgG | Jackson | Cat#111-545-008 |
| | ImmunoResearch | |
| Alexa Fluor 647 Goat anti-Mouse IgG | Jackson | Cat#115-605-003 |
| | ImmunoResearch | |

Chemicals, peptides, and recombinant proteins

| | | |
|---|---|---|
| DMEM/F12 Medium | Gibco | Cat#11330-057 |
| DMEM Medium | Gibco | Cat#11965092 |
| Fetal Bovine Serum (FBS) | Fisher Scientific | Cat#26140079 |
| Penicillin/Streptomycin | Life Technologies | Cat#15140-122 |
| Geneticin | Gibco | Cat#10131-035 |
| Phosphate Buffered Saline (PBS) No calcium no | Gibco | Cat#14190250 |
| magnesium | | |
| 0.25% Trypsin-EDTA (1X) | Gibco | Cat#25200-056 |
| Poly(ethylene glycol), Bio Ultra, 6,000 (PEG6000) | Millipore Sigma | Cat#81253 |
| Poly-L-lysine solution, mol. Wt. 150,000-300,000, | Millipore Sigma | Cat#P4832 |
| 0.01%, sterile-filtered, BioReagent, suitable for cell | | |
| culture | | |
| KOH | Millipore Sigma | Cat#221473 |
| Ethanol | Fisher Scientific | Cat#BP2818500 |
| PIPES disodium salt | Millipore Sigma | Cat#P3768 |
| HEPES | Promega | Cat#H5302 |
| EGTA | Millipore Sigma | Cat#E4378 |
| MgSO₄ | Millipore Sigma | Cat#M5921 |
| Paraformaldehyde (PFA) 37% | Electron Microscopy | Cat#15714-5 |
| | Services | |
| Triton-X-100 | Millipore Sigma | Cat#T9284 |
| Bovine Serum Albumin (BSA) | Millipore Sigma | Cat#A2153 |
| Tween ®20 | Millipore Sigma | Cat#P2287 |
| Fluoro-Gel mounting medium with TES buffer | Electron Microscopy | Cat#17985-30 |
| | Sciences | |
| Vectashield Vibrance Antifade Mounting Media with | Vector Laboratories | Cat#H-1800 |
| DAPI | | |
| Glutaraldehyde 10% EM Grade | Electron Microscopy | Cat#16120 |
| | Sciences | |
| Paraformaldehyde 16% solution | Electron Microscopy | Cat#15710 |
| | Sciences | |
| Sodium phosphate dibasic | Electron Microscopy | Cat#21182 |
| | Sciences | |
| Sodium phosphate monobasic | Ted Pella INc | Cat#19542 |
| Osmium tetroxide | Electron Microscopy | Cat#19100 |
| | Sciences | |
| Potassium ferrocyanide | Millipore Sigma | Cat#P9387 |
| Ethanol | Decon laboratories | Cat#2701 |
| | INc | |
| Acetone 99.8%, Extra dry Acroseal | Acros Organics | Cat#326800010 |
| EMbed 812/DER 736 kit | Electron Microscopy | Cat#14130 |
| | Sciences | |
| EMbed 812 resin | Electron Microscopy | Cat#14900 |
| | Sciences | |
| Uranyl acetate | Electron Microscopy | Cat#22400 |
| | Sciences | |
| Reynolds lead citrate - Lead Nitrate | Acros Organics | Cat#423855000 |
| Reynolds lead citrate - Sodium Citrate Dihydrate | Chem-Impex | Cat#01265 |
| Reynolds lead citrate - Sodium Hydroxide 1N | Acros Organics | Cat#12460010 |

Experimental models: Cell lines

| | | |
|---|---|---|
| HeLa | ATCC | Cat#CCL-2 |
| HeLa MKLP1-GFP (Douglas et al 2010) | Gift from Mishima lab | N/A |

Other

| | | |
|---|---|---|
| T75 flask | Fisher Scientific | Cat#FB012937 |
| T175 flask | Fisher Scientific | Cat#12-556-011 |
| 50 mL tubes | Falcon | Cat#352098 |
| Steriflip Tube top filter unit | Sigma Millipore | Cat#SE1M179M6 |
| Circular Cover Glass, #1.5, Diameter = 18 mm, | Electron Microscopy | Cat#101413-518 |
| Thickness = 0.16-0.19 mm | Sciences | |
| Glass slide | Fisher Scientific | Cat#12-550-413 |
| 12 well tissue culture plate | Fablab | Cat#FL7111 |
| 1.5 ml tube | Fisher | Cat#05-408-129 |
| Swinging Bucket | Eppendorf | Cat#022638866 |
| Revolve Microscope | Echo | RVL2-X |
| Confocal Microscope | Nikon | A1R-Si+ |
| Structured Illumination (SIM) Microscope | Nikon | ECLIPSE-Ti-E |
| Ultramicrotome | Reichert-Jung | Ultracut E |
| Formvar coated 2 × 1 mm slot Cu grids | Electron Microscopy | Cat#FF2010-Cu |
| | Sciences | |
| Transmission Electron Microscope (TEM) | Philips | CM120 |
| TEM Digital Camera | AMT | Biosprint 12 |

### Materials and Equipment

Medium and solutions are filter-sterilized through a membrane of 0.2 mm or smaller pore size.

HeLa-CCL-2 and HeLa-MKLP1-GFP culture medium

| | Final | | |
|---|---|---|---|
| Reagent | concentration | Volume/Mass | |
| DMEM/F12 medium | N/A | 445 | mL |
| FBS | 10% | 50 | mL |
| Penicillin/Streptomycin | 1% | 5 | mL |
| Geneticin - for MKLP1-GFP culture | 750 mg/mL | 15 | mL/mL |

Store at 2-8° C. for up to 4 weeks.

PHEM buffer

| Reagent | Final concentration | Volume/Mass | |
|---|---|---|---|
| Sterile distilled water | N/A | up to 500 mL | |
| PIPES | 60 mM | 9.07 | g |
| HEPES | 25 mM | 3.25 | g |
| EGTA | 10 mM | 1.9 | g |
| MgSO4 | 4 mM | 495 | mg |

| | | |
|---|---|---|
| 2M KOH (adjust pH 7.0) | N/A | N/A |

Store at 2-8° C. for up to 1 year.

PHEM-paraformaldehyde (PFA)

| Reagent | Final concentration | Volume/Mass | |
|---|---|---|---|
| PHEM buffer | N/A | 44.85 | mL |
| PFA (37%) | 3.7% (v/v) | 5 | mL |
| Triton-X-100 | 0.3% (v/v) | 150 | mL |

Store at −20° C. for up to 4 weeks.

Sodium phosphate
(PB)

| Reagent | Final concentration | Volume/Mass | |
|---|---|---|---|
| Sodium Phosphate monobasic | 0.019M | 227.96 | mg |
| Sodium Phosphate dibasic | 0.081M | 2.17 | g |

| | | |
|---|---|---|
| Sterile distilled water | N/A | Up to 100 mL |

Store at 2-8° C. for up to 1 year.

Immersion-fix sodium phosphate solution

| Reagent | Final concentration | Volume/Mass |
|---|---|---|
| Sodium phosphate buffer (PB) | 0.1M | 6.25 mL |
| PFA (16%) | 2% (v/v) | 1.25 mL |
| Glutaraldehyde (10%) | 2.5% (v/v) | 2.5 mL |

Store at 2-8° C. for up to 1 year.

Post-fix sodium phosphate solution

| Reagent | Final concentration | Volume/Mass |
|---|---|---|
| Sodium phosphate buffer (PB) | 0.1M | Up to 100 mL |
| Osmium tetroxide | 1% | 1 g |
| Potassium ferrocyanide | 1% | 1 g |

Store at 2-8° C. for up to 1 year.

For TEM microscopy, an ultramicrotome (Reichert-Jung, Ultracut E), and Formvar coated 2 3 1 mm slot Cu grids (Electron Microscopy Sciences, Cat. no. FF2010-Cu) are required.
PHEM blocking buffer: add 1.5 g BSA (3% w/v) to the PHEM buffer in a final volume of 50 mL. Store at 2-80° C. for up to 1 year.
30% PEG (w/v): add 15 g PEG6000 to sterile phosphate-buffered saline (PBS) to a final volume of 50 mL.

Store at 2-8° C. for up to 6 months.
PBS-T: add 1 mL of Tween 20 (0.2% [v/v]) to 499 mL of PBS.

Store at 16-25° C. for up to 1 year.

### Step-by-Step Method Details

### HeLa-CCL-2 and HeLa-MKLP1-GFP Cell Passaging and Maintenance

### Timing: Up to 7 Days

This protocol is optimized for isolating large EVs or MBRs from cell culture medium. The cells should be in the active growth phase before plating into T175 flasks for medium collection and MBR isolation (steps 8-10 followed by either steps 11-13 or steps 14-16). HeLa-CCL-2 and HeLa-MKLP1-GFP cells are cultured similarly but may proliferate at differing rates.
1. Passage the cells when they are approximately 70% confluent (3-4 3 10⁶ cells).
2. Remove medium, add 0.25% Trypsin-EDTA, and incubate at 37° C. for 5-10 min until cells detach.
3. Pipette the cells up and down to obtain a single-cell suspension.
   a. Mix resuspended cells with an equal volume of DMEM/F12 medium.
   b. Centrifuge at 300 3 g for 5 min.
   c. Remove supernatant and resuspend in DMEM/F12 medium.
4. Split cells 1:5 (6-8×10⁵ cells) into a T75 flask in DMEM/F12 medium.

Note: We suggest cells are split when they reach 60%-70% confluency to ensure they remain in the active growth phase with high levels of mitosis. Geneticin should be added to the MKLP1-GFP medium for one passage every few weeks to maintain selection. MKLP1-GFP HeLa cells have a geneticin resistant cassette linked to MKLP1-GFP. The addition of geneticin antibiotic will eliminate cells without the MKLP1-GFP gene and maintain a culture of MKLP1-GFP positive cells.

### HeLa-CCL-2 and HeLa-MKLP1-GFP Cell Culture on Coverslips

### Timing: 1-3 Days

To visualize cells and MBRs together, HeLa-CCL-2 and HeLa-MKLP1-GFP cells can be cultured on coverslips and characterized by immunofluorescence or an assay of choice. MKLP1-GFP cells should be in a non-selection medium (i.e., without geneticin).
5. Plate cells on poly-L-lysine coated coverslips in a 12-well plate in 1 mL DMEM/F12 medium (3-4×10⁴ cells per well).
6. Incubate the cells until they reach 60%-70% confluency.
7. Characterize the cells using immunofluorescence (steps 41-49) or an assay of choice.

Note: We suggest cells are fixed when they reach a confluency of 60%-70% to ensure the cells remain in the active growth phase with high levels of mitosis, enabling the detection of MBRs.

### Collection of HeLa-CCL-2 and HeLa-MKLP1-GFP Medium

### Timing: 3-6 Days

Cells should be 60%-90% confluent before harvesting medium for MBR isolation. HeLa-CCL-2 and HeLa-MKLP1-GFP cells are cultured similarly but may proliferate at differing rates. MKLP1-GFP cells should be in a non-selection medium (i.e., without geneticin) for collection.
8. Plate HeLa cells (6-8 3 10⁵ cells per flask) in T175 flasks in 20 mL DMEM/F12 medium.
9. Once cells are approximately 75% confluent (6-8 3 10⁶ cells), vigorously shake the flask from side to side 10 times to detach as many midbodies from the cells as possible.
   a. Harvest the medium into a 50 ml conical tube.
   b. Centrifuge the medium at 1000 3 g for 10 min to remove dead cell debris.
10. Carefully remove the supernatant (cleared conditioned medium) and transfer to a new 50 mL conical tube.

Note: If the medium turns yellow, indicating a buildup of waste products from the cells, replace it with fresh medium and incubate for at least 24 h before harvesting. If cells are cultured in 150 mm dishes rather than T175 flasks (step 8), shaking the dish cannot be per-formed easily without medium loss. Therefore, vigorous pipetting of the medium over the sur-face of the dish multiple times should be performed to dislodge the MBRs.

CRITICAL: Cells should be cultured in T175 flasks for at least 2 days and no longer than 5 days before collecting medium. This ensures that the cells have had enough time to un-dergo at least 2 cycles of mitosis to produce MBRs, while also ensuring that the cells are not either overconfluent, which could introduce dead cell debris to the medium, or senescent and not proliferating, therefore producing no MBRs (see problem 1).

### Option 1: 1.5% PEG6000

### Timing: 1 Day

Cleared conditioned medium from step 10 can be used to isolate MBRs/large EVs with 1.5% PEG6000 which precipitates the MBRs/large EVs from the medium, allowing them to be centrifuged and concentrated in a smaller volume.
11. To the cleared conditioned medium from step 10 add 30% PEG6000 solution to yield a final PEG6000 concentration of 1.5% (v/v), and incubate for 12-18 h at 2-8° C.
12. Centrifuge at 1000 3 g for 10 min at 4° C. to pellet the PEG/MBR.
13. Resuspend the PEG/MBR pellet in 1 mL sterile PBS.

Note: There is a chance that small extracellular vesicles in cell culture medium from FBS might also precipitate, but the proposed isolation protocol uses very low concentrations of PEG6000. PEG6000 was tested on fresh medium containing FBS, no isolated EVs were observed.

Pause Point: The PEG/MBR PBS solution can be used immediately for further analysis, stored at 4° C. for up to 3 weeks or at −80° C. for up to 2 months.

### Option 2: 30 nm Gold Nanoparticle

### Timing: 1 Day

Cleared conditioned medium from step 10 can be used to isolate MBRs/large EVs using 30 nm gold nanoparticles which precipitates the MBRs/large EVs from the medium, allowing them to be centrifuged and concentrated in a smaller volume.
14. To the cleared conditioned medium from step 10 add PEG5000 coated 30 nm gold particles solution to yield a final concentration of 0.02% (v/v), and incubate for 12-18 h at 2-8° C.
15. Centrifuge at 1000 3 g for 10 min at 4° C. to pellet the gold/MBR.
16. Resuspend the gold/MBR pellet in 1 mL sterile PBS.

Note: There is a chance that small extracellular vesicles in cell culture medium from FBS might also precipitate, but the proposed isolation protocol uses very low concentrations of gold nanoparticles to ensure that small exosomes and other impurities are not precipitated. When the gold nanoparticles were tested on fresh medium containing FBS, no isolated EVs were observed.

Pause Point: The gold/MBR PBS solution from step 16 can be used immediately for further analysis, stored at 4° C. for up to 3 weeks or at −80° C. for up to 2 months.

### MBR Fixation for Transmission Electron Microscopic (TEM) Imaging

### Timing: 2-3

MBR size and structure can be studied using electron microscopy.
17. Centrifuge the PEG/MBR PBS solution from step 13 or gold/MBR PBS solution from step 16 at 1000 3 g for 10 min at 4° C. to pellet the MBRs.
   a. All subsequent steps for TEM are performed on the PEG/MBR or gold/MBR pellet.
   b. If the pellet can no longer be visualized, centrifuge at 1000 3 g for 10 min.
18. Immersion-fix the MBR pellet in immersion fix sodium phosphate solution for 2 h at 20-25° C.
19. Rinse the MBR pellet five times for 5 min in PB.
20. Post-fix the MBR pellet in post fix sodium phosphate solution for 1 h at 20-25° C.
21. Rinse five times for 5 min in PB.
22. Dehydrate the MBR pellet in a graded ethanol series (35%, 50%, 70%, 80% and 90%) diluted in dH₂O for 5 min each at 20-25° C.
23. Dehydrate the MBR pellet in 95% ethanol diluted in dH₂O for 10 min at 20-25° C.
24. Dehydrate the MBR pellet in 100% ethanol twice for 10 min at 20-25° C.
25. Dehydrate the MBR pellet in dry acetone (AC) twice for 7 min at 20-25° C.
26. Incubate the dehydrated MBR pellet with 10% EMbed 812 in AC for 60 min at 20-25° C.
27. Incubate the dehydrated MBR pellet with 25% EMbed 812 in AC for 60 min at 20-25° C.
28. Incubate the dehydrated MBR pellet with 50% EMbed 812 in AC for 12-18 h at 20-25° C.
29. Incubate the dehydrated MBR pellet with 75% EMbed 812 in AC for 60 min at 20-25° C.
30. Incubate the dehydrated MBR pellet with 100% EMbed 812 for 45 min at 60° C.
31. Incubate the MBR pellet in fresh EMbed 812 for 48 h at 60° C. for embedding and polymerization.
32. Section the MBR pellet on an ultramicrotome into 100-nm sections.
33. Collect the sections on 2 3 1 mm slot copper grids.
34. Post-stain with 8% uranyl acetate in 50% ethanol for 15 min at 60° C.
35. Post-stain with Reynolds lead citrate for 10 min at 60° C.
36. Image sections at 80 kV, 40,0003 magnification with a transmission electron microscope.

TABLE 1
Antibodies for immunofluorescence

| Antibody | Dilution | Supplier |
|---|---|---|
| Primary antibody | | |
| Anti-MKLP1 | 1:2000 | Novus Biologicals |
| Anti-CD9 | 1:500 | Santa Cruz Biotechnology |
| Secondary antibody | | |
| Alexa Fluor 488 goat anti-mouse | 1:500 | Jackson ImmunoResearch |
| Alexa Fluor 647 goat anti-rabbit | 1:500 | Jackson ImmunoResearch |
| Alexa Fluor 555 anti-a-tubulin | 1:500 | Millipore Sigma |

### MBR Attachment to Poly-L-Lysine Coated Coverslip

### Timing: 30 Min

MBRs from step 13 or 16 can be attached to poly-L-lysine-coated coverslips using the enhanced electrostatic interaction between the cell membrane and the poly-L-lysine for characterization by immunofluorescence or an assay of choice. Without the poly-L-lysine coating the MBRs do not attach well to glass and can be lost from the coverslip (see problem 1).
37. Using forceps place one poly-L-lysine coated coverslip into each well of a 12-well plate.
38. Add 500-1000 mL of PEG/medium solution to each coverslip.
39. Centrifuge the plate at 1000 3 g for 10 min in a tabletop centrifuge using a swinging bucket.
40. Carefully aspirate the supernatant without disturbing the coverslip.

Note: The coverslips can be used for analysis pre-fixation and immunofluorescent staining after fixation.

### Characterizing MBRs: Immunofluorescent Antibody Staining

### Timing: ˜2 Days

Midbody remnants (MBRs) are released when a cell undergoes mitosis. MBRs or large extracellular vesicles (EV) can be distinguished from other EVs by the presence of MKLP1, a midbody marker protein (Patel et al., submitted). MBRs, either isolated or attached to cells, can be characterized by immunofluorescent staining. Here, the MBRs were stained for MKLP1,¹⁻⁴ a widely used marker for midbodies, and CD9, a tetraspanin protein found on the surface of all classes of extracellular vesicles.⁵⁻⁷
41. Fix the MBR coverslips from step 40 or cell and MBR coverslips from step 7 with PHEM-PFA for 15 min at 20-25° C.
42. Wash the fixed coverslips three times for 5 min with PBS.
43. Block the coverslips with PHEM blocking buffer for 45-60 min at 37° C. or 2 h at 20-25° C.
44. Incubate coverslips with primary antibody (Table 1) diluted in PHEM blocking buffer for 12-18 h at 4° C.
45. Wash coverslips three times for 5 min with PBS-T.
46. Incubate the coverslips with secondary antibody (Table 1) diluted in PHEM blocking buffer for 30 min at 37° C., 1 h at 20-25° C., or 12-18 h at 4° C.
a. If required, fluorophore-conjugated antibodies can be added along with the secondary antibodies.
47. Wash the coverslips four times for 5 min with PBS-T.
48. Mount the coverslips onto slides with a drop of Fluoro-Gel mounting medium for the isolated MBRs or Vectashield mounting medium for cells with MBRs.
49. Image the MBRs with a 203 objective, or higher, as required.

Note: We used a Nikon Structured illuminated microscope (N-SIM) with 1003 objective and an Echo Revolve microscope with a 203 objective to obtain images of MBRs.

### Characterizing MBRs: Immunofluorescent Imaging

### Timing: ˜2 Days

MBRs obtained from step 13 or 16 from HeLa-MKLP1-GFP cells can be directly characterized by immunofluorescence imaging because the MBRs are GFP-positive.
50. Fix the MBR-containing coverslips with PHEM-PFA for 15 min at 20-25° C.
51. Wash the fixed coverslips three times for 5 min with PBS.
52. Mount the coverslips onto slides with a drop of Fluoro-Gel mounting medium.
53. Image the MBRs with a 203 objective, or higher, as required.

Note: If required, the MKLP1-GFP MBRs can also be stained with CD9, following steps 44-47. If there are a large number of small vesicles that are CD9 positive (exosome marker) and MKLP1-GFP negative please refer to problem 2. We used a Nikon Structured illuminated microscope (N-SIM) with a 1003 objective and an Echo Revolve microscope with a 203 objective to obtain images of MBRs. Please refer to problem 1 if there are low numbers of MKLP1 positive vesicles visible.

### Expected Outcomes

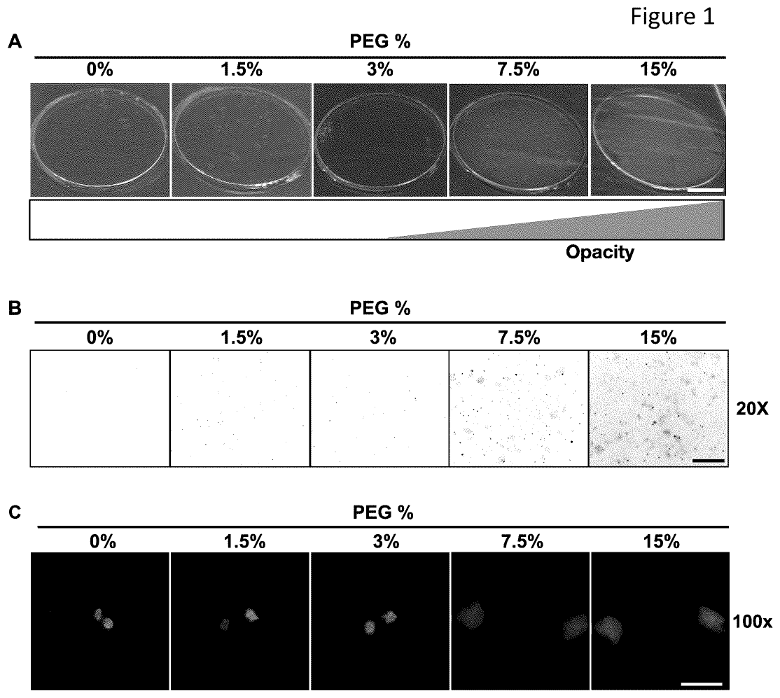

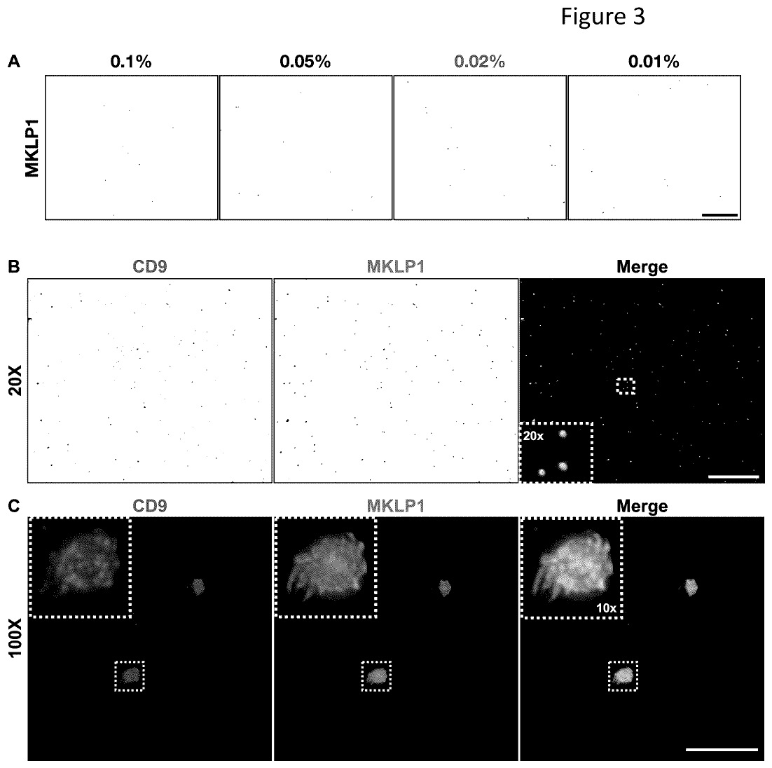

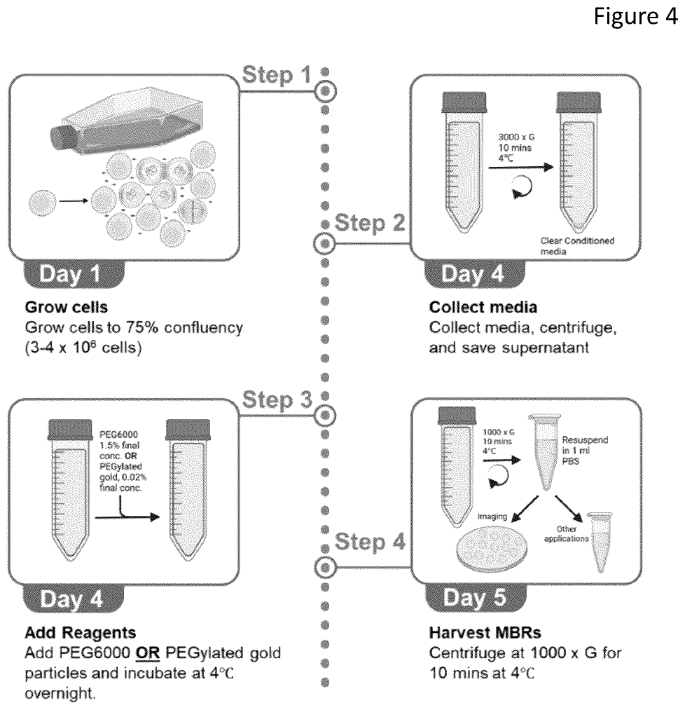

The protocol presented here is adapted and optimized from previously published protocols for isolating extracellular vesicles (EVs) including exosomes.⁸⁻¹¹ PEG6000 has been used to precipitate exosomes and large EVs. Modifying the PEG concentration can effectively isolate large extracellular vesicles or MBRs with a uniform membrane structure (FIG. 1). PEG coated gold and iron (II,III) oxide nanoparticles have been used to isolate exosomes and extracellular vesicles.^{6,9} Modifying the con-centration of the gold nanoparticles can effectively isolate large extracellular vesicles or MBRs from cell culture media (FIG. 3).

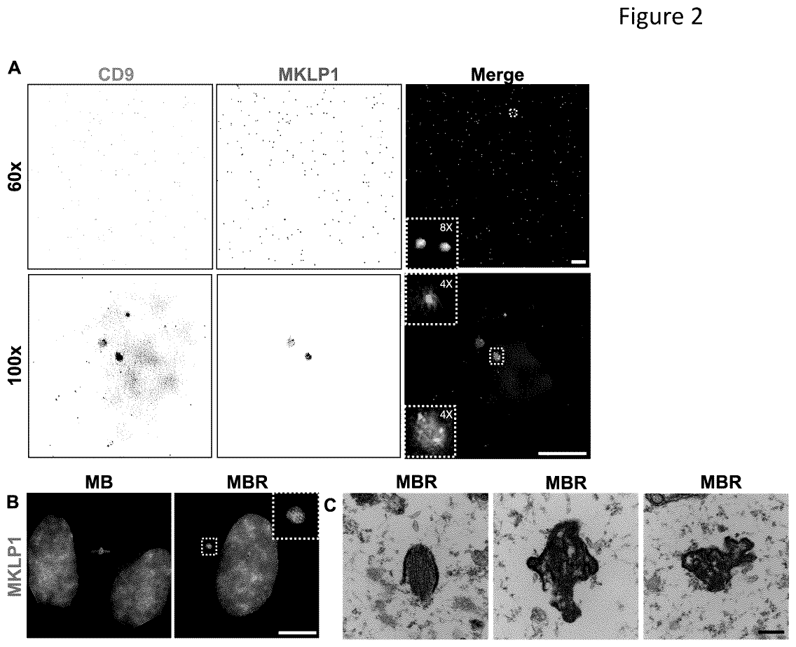

High PEG6000 concentrations (8%, currently used in the field) distort large EVs and MBRs, giving them a swollen, deformed appearance (FIGS. 1B and 1C). With 1.5% PEG6000, the large EVs/MBRs are smaller and circular and have a brighter MKLP1-GFP intensity (FIG. 1C), which mimics those observed in midbodies (MB) and MBRs in cells (FIG. 2B). Higher PEG concentrations inter-fere with cell membrane fluidity by modifying lipids on the surface membrane.¹¹⁻¹⁵ Hence, higher PEG concentrations can compromise the structure and activity of the MBR surface membrane, making it appear dull and misshapen (FIG. 1C). PEG6000 is a hydrophilic polymer which dissolves in water based solution, this reduces the solubility of larger particles in solution, such as large EVs/MBRs.⁸ Therefore, these particles precipitate and can be pelleted when centrifuged at low speeds (1000 3 g for 10 min). A PEG6000 concentration of 1.5% is too low to precipitate small EVs or exosomes, which remain in solution when centrifuged at low speeds. Thus, the PEG concentration correlates negatively with the size of particles precipitated and higher PEG concentrations foster smaller off-target extracellular vesicles.⁸

There are two proposed mechanisms by which PEG-coated gold nanoparticles may function in isolating MBRs. Firstly, PEG-coated gold nanoparticles may weigh down exosomes in solution, making them easier to pellet at low-speed centrifugation.⁹ Secondly, the branched chains of PEG on PEG-coated Fe₃O₄ nanoparticles can increase their surface-to-volume ratio and trap small proteins and impurities, enabling pure exosomes to be pelleted during centrifugation.⁶

To show that the isolated particles are large EVs or MBRs, the isolated particles can be labeled with CD9 and MKLP1 (FIG. 2A). This staining shows an overlap between CD9 and MKLP1; however, the CD9 channel has a cloudy background that is missing from the MKLP1 channel. TEM confirms that the particles isolated by the current protocol are MBRs with a dark core formed by an electron-dense region that the electrons from the TEM cannot pass through (FIG. 2C). The images are similar to previously published reports,^{3,13,16-18} which showed the midbody and MBR as a tubulin-rich, electron-dense dark region.

We tested different concentrations of PEG-coated gold nanoparticles in clear conditioned media and compared their results (FIG. 3A). A concentration of 0.02% v/v of PEG-coated gold nanoparticles is sufficient to yield a high concentration of MBRs. We identified isolated MBRs using MKLP1, a highly specific MBR marker, through HeLa CCL-2 MBR staining and the use of the MKLP1-GFP HeLa cell line (FIGS. 3B and 3C).

Skop Lab 1.5% PEG6000 and Skop lab 30 nm gold nanoparticles are both effective protocols of producing a highly concentrated solution of large EVs/MBRs from cell culture medium. They both isolate particles that maintain biological properties, such as translation. The advantage of Skop Lab 1.5% PEG6000 is that it is very cost effective and isolates a larger number of MBRs than the Skop Lab 30 nm gold nanoparticle protocol. However, the Skop Lab 30 nm gold nanoparticle protocol uses 0.02% (v/v) of reagent versus PEG6000 which uses 1.5% (v/v) of reagent. This reduces the amount of contaminating particles within the final large EV/MBR solution (Patel et al.).

The purpose of this study was to improve MBR or large EV isolation protocols by using MKLP1 to identify large EVs/MBRs (1-2 mm) from smaller EVs or exosomes (30-500 nm) (FIG. 2). Moreover, we wanted to reduce the cost and time required for MBR isolation compared to protocols currently on the market that are used to isolate all sizes of EVs. These assays require access to three types of ultracentrifuges, making the isolation more expensive. The 1.5% PEG6000 and 30 nm gold nanoparticle protocols and use of MKLP1 as a marker for large EVs are more cost-effective and easier to identify large EVs, making these protocols more accessible to all types of laboratories.

### Quantification and Statistical Analysis

MBR isolation by these protocols were repeated a minimum of 25 times, and MBRs (5-20 3 10⁵) were successfully isolated on each occasion from a T75 flask.

### Limitations

Cell confluency may be variable throughout the flask. Therefore, we use an average confluency in the protocol. Differences in seeding cell number and growth time may alter the confluency, which can affect the MBR yield during isolation.

### Troubleshooting

### Problem 1

When viewed by microscopy, there is poor MBR yield with few particles labeled with anti-MKLP1/GFP (related to characterizing the MBRs and MBR attachment to poly-L-lysine coated coverslip).

### Potential Solution

The density of MBRs in the medium may have been low prior to adding the PEG6000 or 30 nm gold nanoparticle solution. To obtain higher numbers of MBRs, ensure the cells are at least 75% confluent before medium collection (Step 9) and shake the flask vigorously to detach MBRs from the cells and flask surface.
The poly-L-lysine coverslips may have been stored incorrectly (i.e., too long), or the poly-L-lysine solution used was expired. Make new coated coverslips with fresh poly-L-lysine solution and repeat attachment of MBRs to coverslips.
When fixing, washing, and staining the coverslips, carefully add and remove the solutions down the side of the well rather than direct pipetting onto the coverslips as shear force could potentially detach the MBRs.

### Problem 2

Many small CD9-positive exosomes are attached to the coverslip alongside the MBRs (related to step 36).

### Potential Solution

When attaching the MBRs onto the coverslips, the plate may be spun at a lower speed of 500 3 g for 10 min, ensuring only the larger MBRs attach to the coverslip and any small exosomes remain suspended in solution.

### REFERENCES

1. Douglas, M.E., Davies, T., Joseph, N., and Mishima, M. (2010). Aurora B and 14-3-3 Coordinately Regulate Clustering of Centralspindlin during Cytokinesis. Curr. Biol. 20, 927-933.
2. Park, S., Dahn, R.D., Kurt, E., Presle, A., VandenHeuvel, K., Moravec, C., Jambhekar, A., Olukoga, O.K., Shepherd, J.D., Echard, A.D., et al. (2022). The Midbody and Midbody Remnant Are Assembly Sites for RNA and Active Translation.
3. Hu, C.-K., Coughlin, M., and Mitchison, T.J. (2012). Midbody assembly and its regulation during cytokinesis. Mol. Biol. Cell 23, 1024-1034.
4. Matuliene, J., and Kuriyama, R. (2002). Kinesin-like Protein CHO1 Is Required for the Formation of Midbody Matrix and the Completion of Cytokinesis in Mammalian Cells. Mol. Biol. Cell 13, 1832-1845.
5. Yoshioka, Y., Konishi, Y., Kosaka, N., Katsuda, T., Kato, T., and Ochiya, T. (2013). Comparative marker analysis of extracellular vesicles in different human cancer types. J. Extracell. Vesicles 2, 20424.
6. Chang, M., Chang, Y.-J., Chao, P.Y., and Yu, Q. (2018). Exosome purification based on PEG-coated Fe3O4 nanoparticles. PLOS One 13, e0199438.
7. Hoshino, A., Kim, H.S., Bojmar, L., Gyan, K.E., Cioffi, M., Hernandez, J., Zambirinis, C.P., Rodrigues, G., Molina, H., Heissel, S., et al. (2020). Extracellular Vesicle and Particle Biomarkers Define Multiple Human Cancers. Cell 182, 1044-1061.e18.
8. Rider, M.A., Hurwitz, S.N., and Meckes, D.G. (2016). ExtraPEG: A Polyethylene Glycol-Based Method for Enrichment of Extracellular Vesicles. Sci. Rep. 6, 23978.
9. Pammi Guru, K.T., Sreeja, J.S., Dharmapal, D., Sengupta, S., and Basu, P.K. (2022). Novel Gold Nanoparticle-Based Quick Small-Exosome Isolation Technique from Serum Sample at a Low Centrifugal Force. Nanomaterials-basel 12, 1660.
10. Garcia-Romero, N., Madurga, R., Rackov, G., Palaci'n-Aliana, I., Nu'n'ez-Torres, R., Asensi-Puig, A., Carrio'n-Navarro, J., Esteban-Rubio, S., Peinado, H., Gonza\acute{ }lez-Neira, A., et al. (2019). Polyethylene glycol improves current methods for circulating extracellular vesicle-derived DNA isolation. J. Transl. Med. 17, 75.
11. Lee, S.-S., Won, J.-H., Lim, G.J., Han, J., Lec, J.Y., Cho, K.-O., and Bac, Y.-K. (2019). A novel population of extracellular vesicles smaller than exosomes promotes cell proliferation. Cell Commun. Signal. 17, 95.
12. Melikishvili, S., Poturnayova, A., Ionov, M., Bryszewska, M., Vary, T., Cirak, J., Munoz-Fernandez, M.A., Gomez-Ramirez, R., de la Mata, F.J., and Hianik, T. (2016). The effect of polyethylene glycol-modified lipids on the interaction of HIV-1 derived peptide-dendrimer complexes with lipid membranes. Biochim. Biophys. Acta 1858, 3005-3016.
13. Ettinger, A.W., Wilsch-Brauninger, M., Marzesco, A.-M., Bickle, M., Lohmann, A., Maliga, Z., Karbanova, J., Corbeil, D., Hyman, A.A., and Huttner, W.B. (2011). Proliferating versus differentiating stem and cancer cells exhibit distinct midbody-release behaviour. Nat. Commun. 2, 503.
14. Chen, C.-T., Ettinger, A.W., Huttner, W.B., and Doxsey, S.J. (2013). Resurrecting remnants: the lives of post-mitotic midbodies. Trends Cell Biol. 23, 118-128.
15. Dutheil, D., Underhaug Gjerde, A., Petit-Paris, I., Mauco, G., and Holmsen, H. (2009). Polyethylene glycols interact with membrane glycerophospholipids: is this part of their mechanism for hypothermic graft protection? J. Chem. Biol. 2, 39-49.
16. Skop, A.R., Liu, H., Yates, J., Meyer, B.J., and Heald, R. (2004). Dissection of the Mammalian Midbody Proteome Reveals Conserved Cytokinesis Mechanisms. Science 305, 61-66.
17. Crowell, E.F., Gaffuri, A.-L., Gayraud-Morel, B., Tajbakhsh, S., and Echard, A. (2014). Engulfment of the midbody remnant after cytokinesis in mammalian cells. J. Cell Sci. 127, 3840-3851.
18. Fremont, S., and Echard, A. (2017). Studying cytokinesis and midbody remnants using correlative light/scanning EM. Methods Cell Biol. 137, 239-251.

### Example 2—The Biogenesis of Large Extracellular Vesicles Occurs in Mitosis

### Summary:

This study challenges the long-held belief that large extracellular vesicles (EVs) arise from cells in interphase. Through multiple common and commercial isolation methods and immunofluorescent studies on HeLa cells, we found that canonical EV markers localize to the spindle midzone and midbody during mitosis, subsequently remaining in the midbody remnant (MBR). All canonical EV markers co-localized with MKLP1, a midbody and MBR marker, suggesting that this class of large EVs is generated during mitosis. Puromycin assays revealed that all commonly used EV isolation methods have been isolating translating MBRs. Chemical and genetic blocks for mitosis significantly decreased the formation of all EV sizes, implying that mitotic cells generate large quantities of EVs. The findings suggest that large EVs originate during mitosis, are midbody remnants, and that MKLP1 and translation activity are unique markers for this EV class.

### Significance:

Extracellular vesicles (EVs) play a crucial role in cell-cell communication, but the biogenesis of large EVs has remained elusive. This study addresses the gap in understanding the origin of these large EVs.
The key findings reveal that the biogenesis of large EVs primarily occurs during mitosis, and these vesicles are midbody remnants (MBRs) containing translation machinery. Importantly, all commonly used EV isolation methods isolated MBRs, indicating that previous EV research may have contained mitotic EVs.
These findings are significant as they identified a unique class of large, translating EVs (MBRs) with potential implications for cancer diagnosis and drug delivery. The findings suggest that MBRs could serve as diagnostic markers for cancer and be engineered for therapeutic cargo delivery during mitosis, influencing future EV research and therapeutic development.

Extracellular vesicles (EVs) are particles that are released from almost every type of cell, are bound by a lipid bilayer, and function in cell-to-cell communication (Villa 2019, Yáñez-Mó 2015, Pol 2012, Xie 2022, Hanayama 2020, Janas 2016, Jeppesen 2023, Tetta 2013, Xu 2018, Cheng 2023, Colombo 2014). Depending on the cell type they originate from, EVs can contain RNA, DNA, lipids, metabolites, and cell surface proteins (Dellar et al., 2022; Qian et al., 2022). There is a strong bias towards RNA molecules, including mRNA, microRNA, ribosomal RNA (rRNA), and long non-coding RNA (IncRNA) (Zhao 2020, Hinger 2018, Mathivanan 2010,Lischnig 2022). RNA is transferred to recipient cells and can contribute to many functions, including repair of damaged cells, cancer progression, and neurodegeneration (Valadi et al., 2007; Chen et al., 2016; Hinger et al., 2018; O'Brien et al., 2018; Li et al., 2019; Makarova et al., 2021). Despite extensive research in the field for over a decade, the biogenesis of large EVs remains surprisingly unclear (Gurung 2021, Gurunathan 2021, Xie 2022, Latifkar 2019, Margolis 2019, Suwakulsiri 2023, Akers 2013, Beshbishy 2020).

EVs are currently classified into four distinct groups based on size: 1) exosomes or small EVs (40-100 nm), 2) microvesicles (100-1000 nm), 3) large EVs (1,000-2,000 nm), and 4) apoptotic bodies (1,000-10,000 nm) (Brennan 2020, Lee 2019, Verweij 2021, Suwakulsiri 2023, Rai 2021). The biogenesis of exosomes is well understood (Kalluri and LeBleu, 2020), and apoptotic bodies are generated by dying cells (Teng 2021). However, the biogenesis of microvesicles and large EVs are less understood. Current understanding suggests they primarily arise from interphase cells by shedding or budding (Gurunathan 2021, Beshbishy 2020, Niel 2018, Margolis 2019, Akers 2013), but methods used to define mitotic EVs were biochemical, density gradients and limited imaging, all of which failed to accurately demonstrate the EVs were derived from mitosis (Karbanová 2024, Suwakulsiri 2023). Many in the EV field recognize that vesicle sizes often overlap, there are no distinct markers for each subtype, and research outcomes are strongly influenced by the isolation methods used (Andreu 2014). These factors lead to uncertainty, especially in the interpretation of results. Despite seeking to standardize terminology, methods, and reporting across studies (Deun et al., 2017; Théry et al., 2018), most researchers have assumed that all EVs originate from interphase cells either by budding (for microvesicles or microparticles) or exocytosis (for exosomes and large EVs) (Doyle 2019, Beshbishy 2020, Gurunathan 2021, Xie 2022, Cheng 2023, Latifkar 2019, Teng 2021, Gandham 2020, Margolis 2019, Akers 2013, Suwakulsiri 2023). However, we surmised that cells are unlikely to generate a 1-2 μm large EVs during interphase due in part to the biophysical and physiological aspects of the biology of Hela cells, which are roughly 18 μm, and stem cells, which are ˜11-12 μm. In addition, the only time a cell normally generates a 1-2 μm vesicle is during mitosis at abscission, which produces the midbody remnant (MBR) (Crowell 2014, Crowell 2013, Peterman 2019, Rai 2021, Addi 2020, Park 2023).

Uncertainty remains in the use of tetraspanins as markers of EVs (Andreu and Yáñez-Mó, 2014a; Mizenko et al., 2021). Tetraspanins are transmembrane proteins that are involved in signaling, are found in all membranes, and are not specific to exosomes, microvesicles/microparticles, large EVs, or apoptotic bodies (Hemler, 2001; Termini and Gillette, 2017). However, a recent proteomic study by Lischnig et al., 2022, isolated EVs from cell culture and classified them into two groups: large EVs (derived by centrifuging at 16,500×g) and small EVs (derived by centrifuging at 118,000×g) (Lischnig 2022). The density of both EV groups was measured, and their protein contents were analyzed by mass spectrometry. The study revealed that both large and small EVs had the same density and were marked by the tetraspanins CD9, CD81, CD63, and Flotillin-1. This suggests that tetraspanins are not ideal for isolation purposes despite being widely used in the field (Andreu 2014). Lischnig et al., 2022 also discovered that large EV proteomes specifically contained large amounts of KIF23, also known as MKLP1, and its effector RACGAP1, which were absent from small EV proteomes (Lischnig 2022), suggesting to us that large EVs are likely of mitotic origin. Equally puzzling, is the presence of KIF23/MKLP1 on Westerns of isolated large EVs with different densities (Suwakulsiri 2023), suggesting that the large microvesicles (also known as microparticles) may be of mitotic origin. Lastly, human colorectal cancer cells were shown to shed large numbers of MKLP1/RACGAP1 positive vesicles versus normal tissue (Rai 2021). Given these three papers, we hypothesized that large EVs, regardless of different densities or sizes, are actually midbody remnants (MBRs). This is due in part because MKLP1/KIF23 and RACGAP1 are canonical markers for the midbody (Kuriyama et al., 2002; Matuliene and Kuriyama, 2002a; Zhu et al., 2005; Hu et al., 2012).

MBRs arise at the end of cytokinesis during a process called abscission, which separates the newly formed daughter cells (Echard 2004, Addi 2020, D'Avino 2016, Bassi 2013, Park 2023, Crowell 2014, Skop 2004). MBRs have been suggested to play post-mitotic roles in cell-cell communication, cell fate, and signaling, which are proposed roles for EVs (Chaigne 2020, Chaigne 2022, Addi 2020, Peterman 2019, Peterman 2019). Historically, isolation of MBRs was performed using ultracentrifugation from synchronized or asynchronized cells (Addi 2020, Peterman 2019, Skop 2004). Identification of midbodies and MBRs is easily accomplished using the centralspindlin complex member MKLP1 as a marker (Zhu et al., 2005; Hu et al., 2012; White and Glotzer, 2012). MKLP1 first localizes to the spindle midzone during anaphase and then to the midbody and MBR. MKLP1 is widely conserved, making it an excellent marker for a variety of cell types (Mishima 2004, Kuriyama 2002, Matuliene 2002, Zhu 2005). More recently, MBRs have been identified as a new class of EVs (Rai 2021); however, we propose that the majority of the large EVs are MBRs.

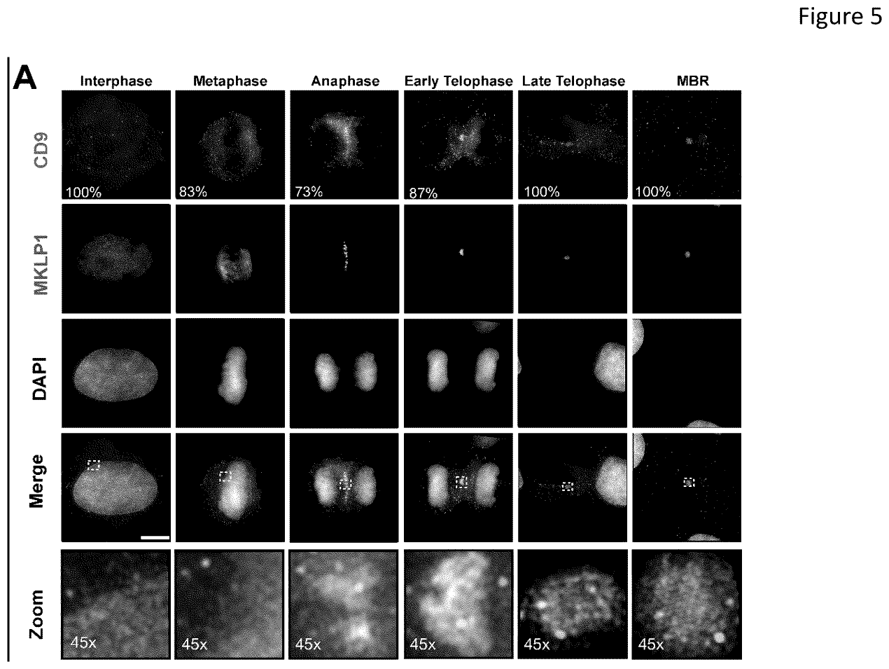

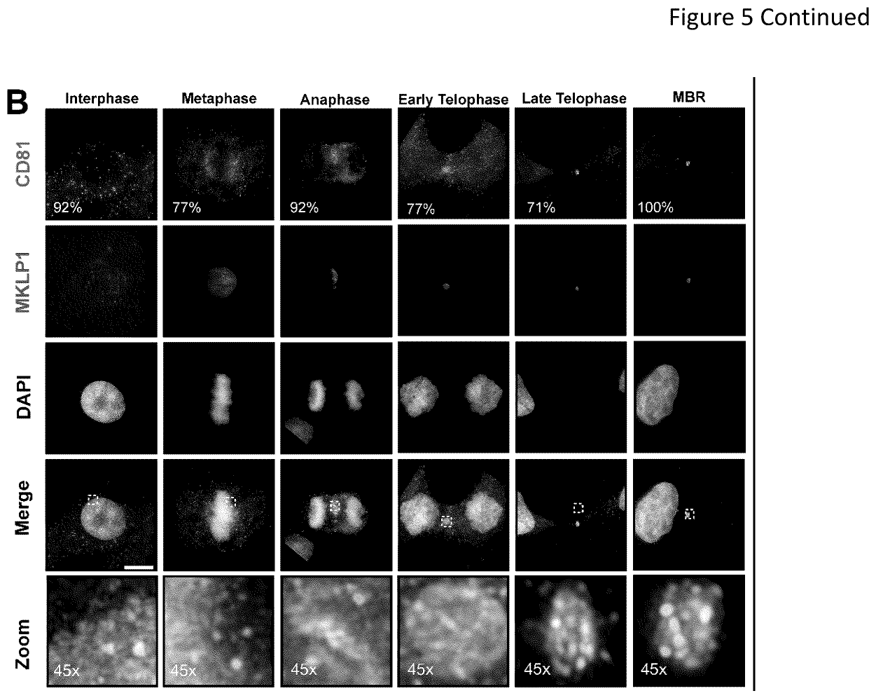

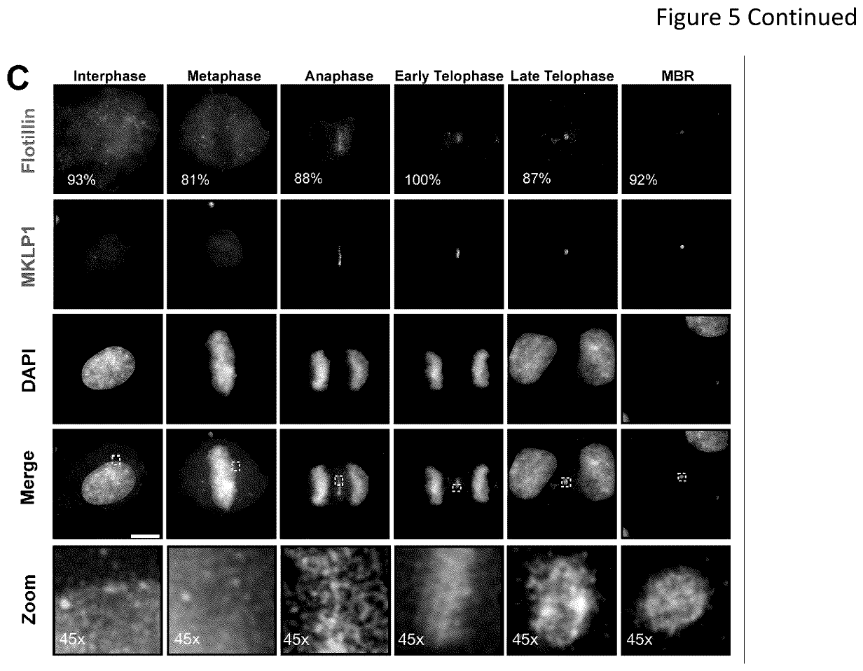

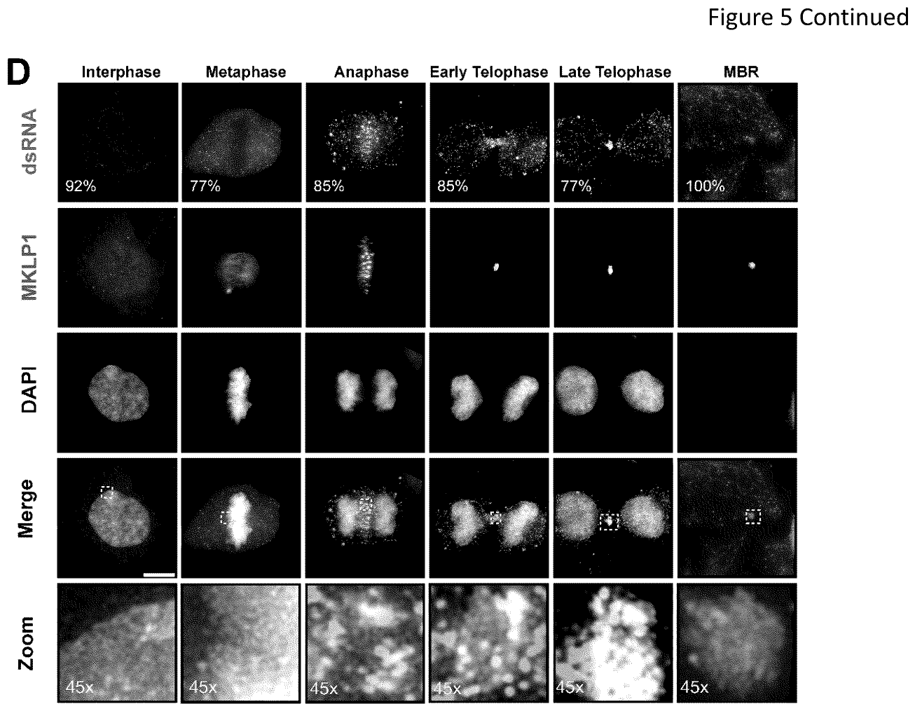

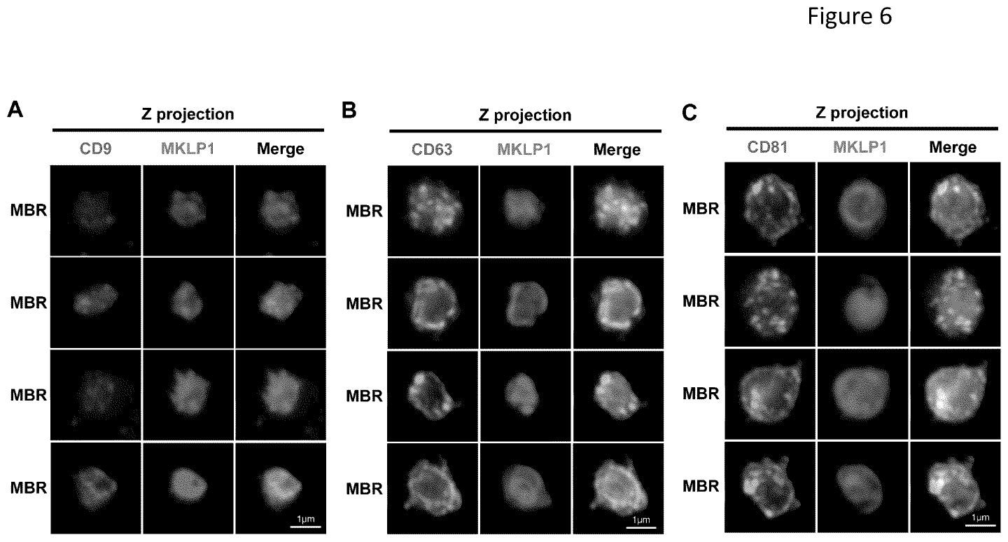

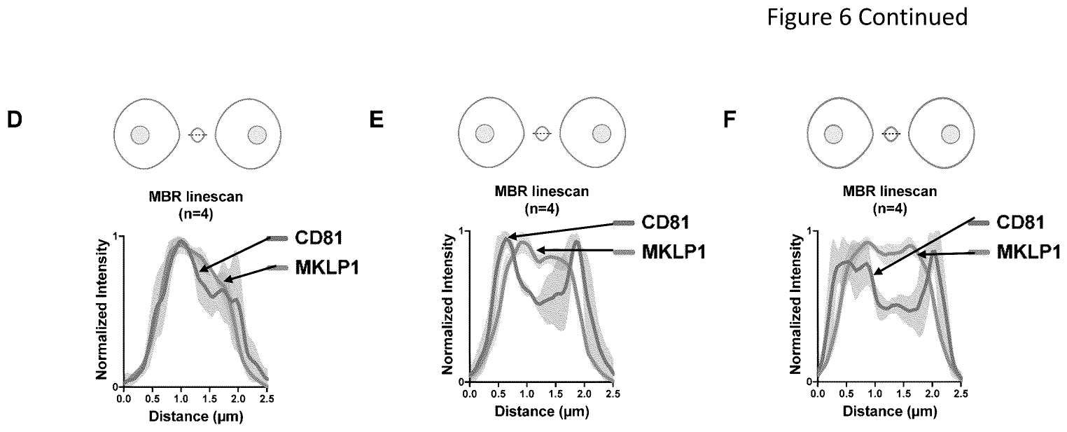

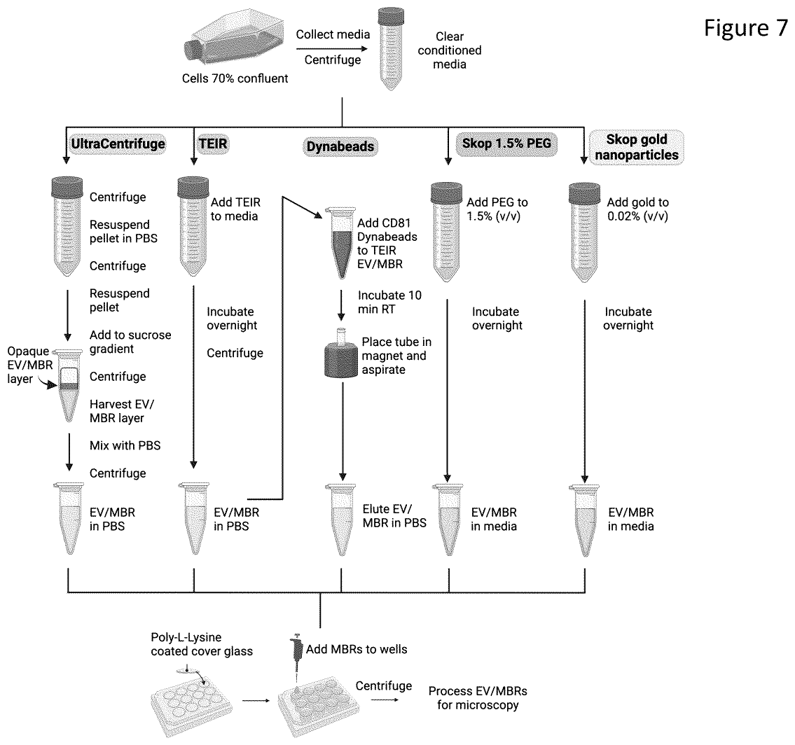

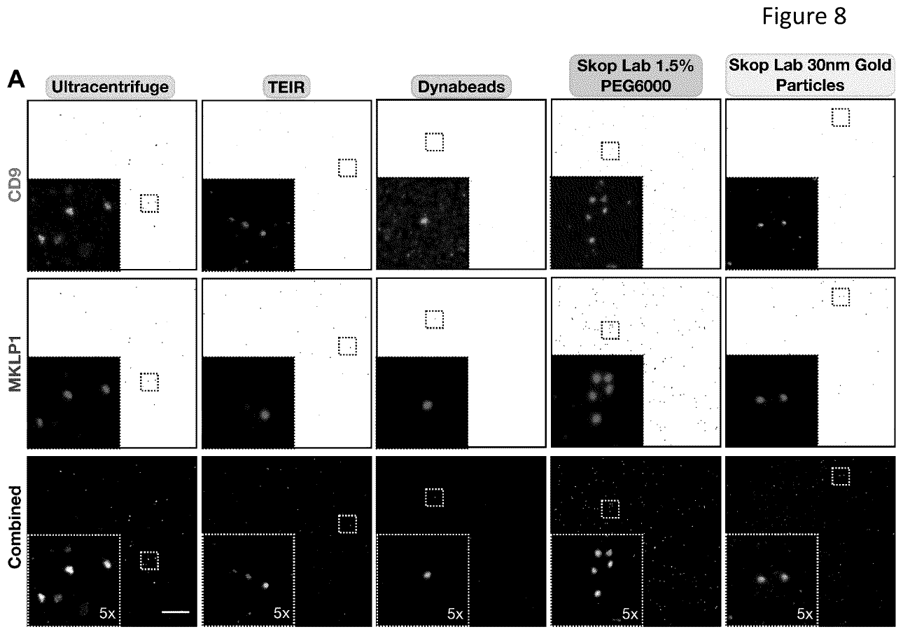

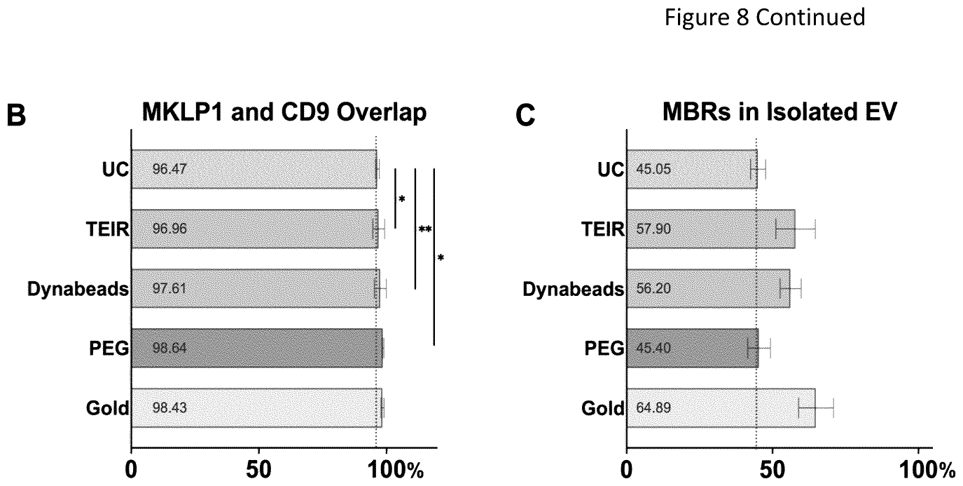

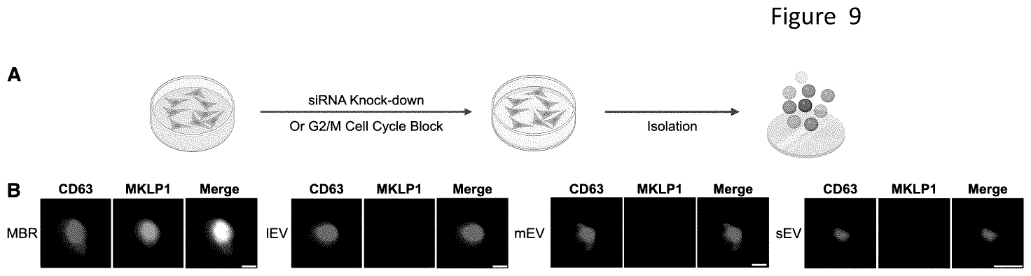

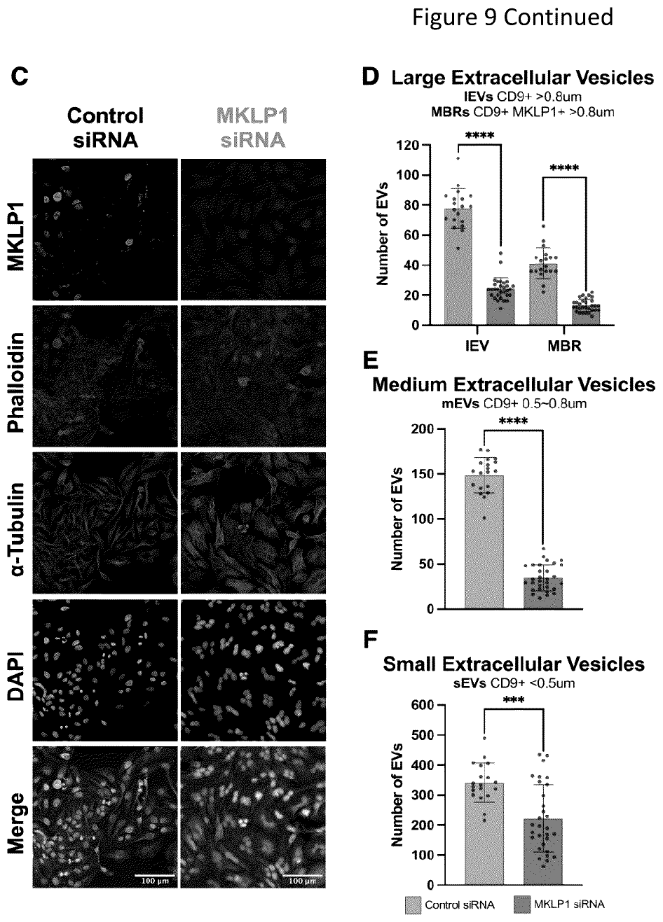

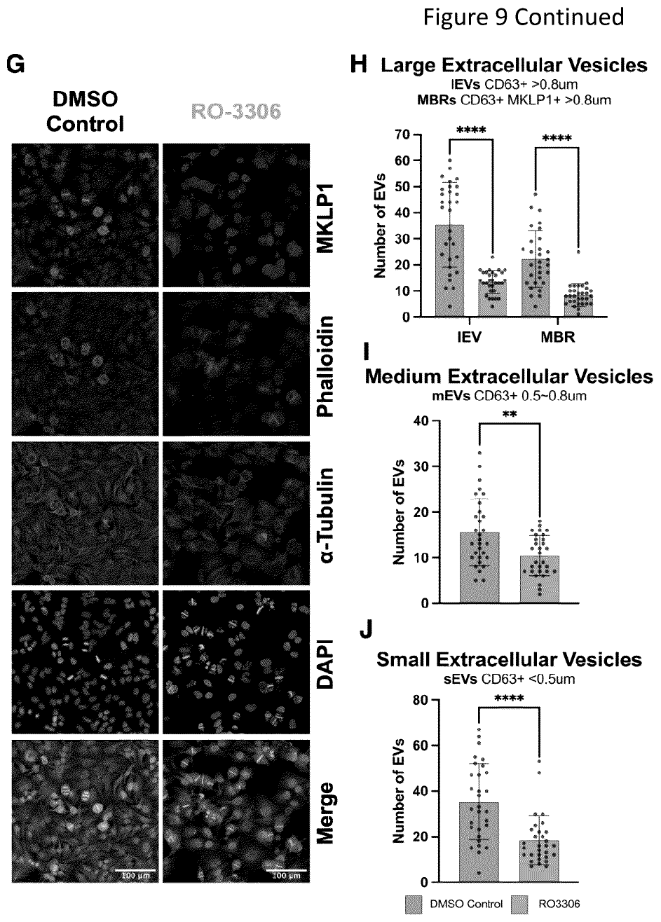

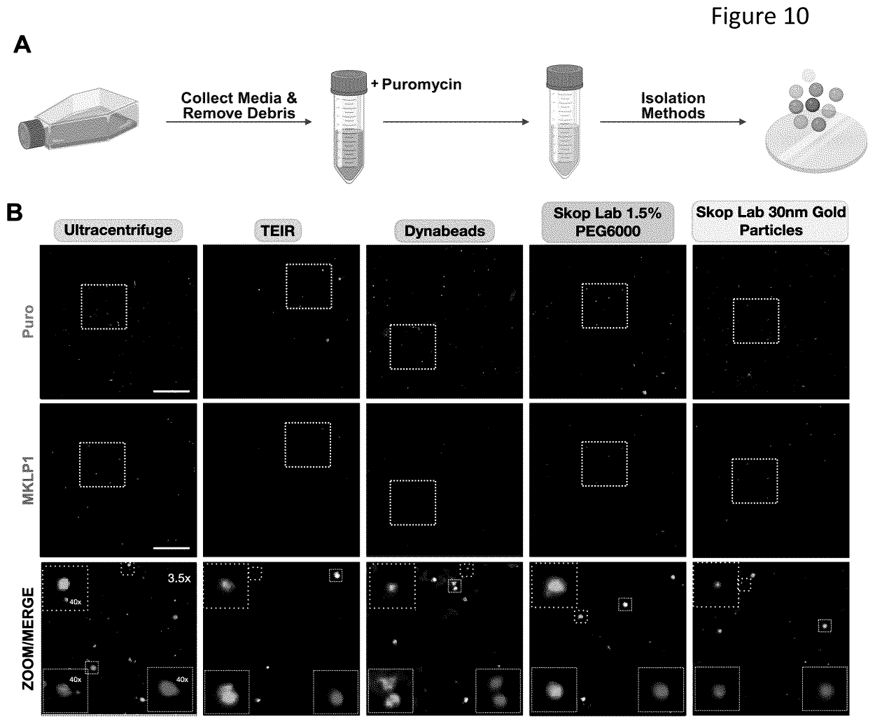

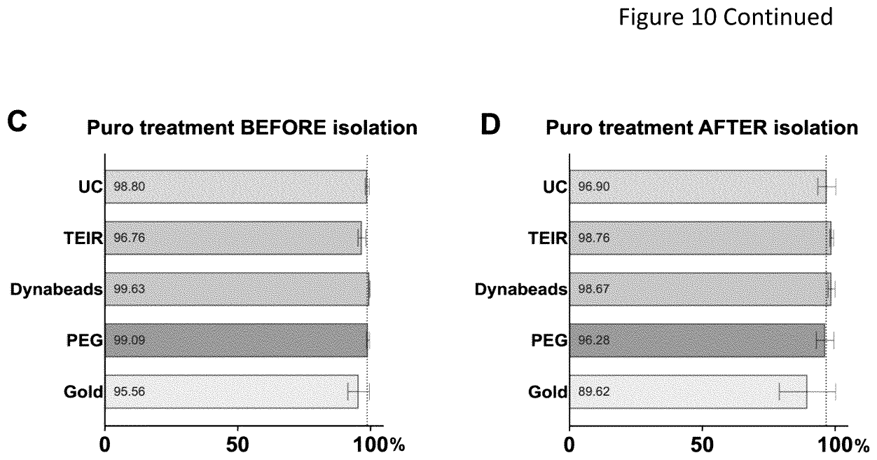

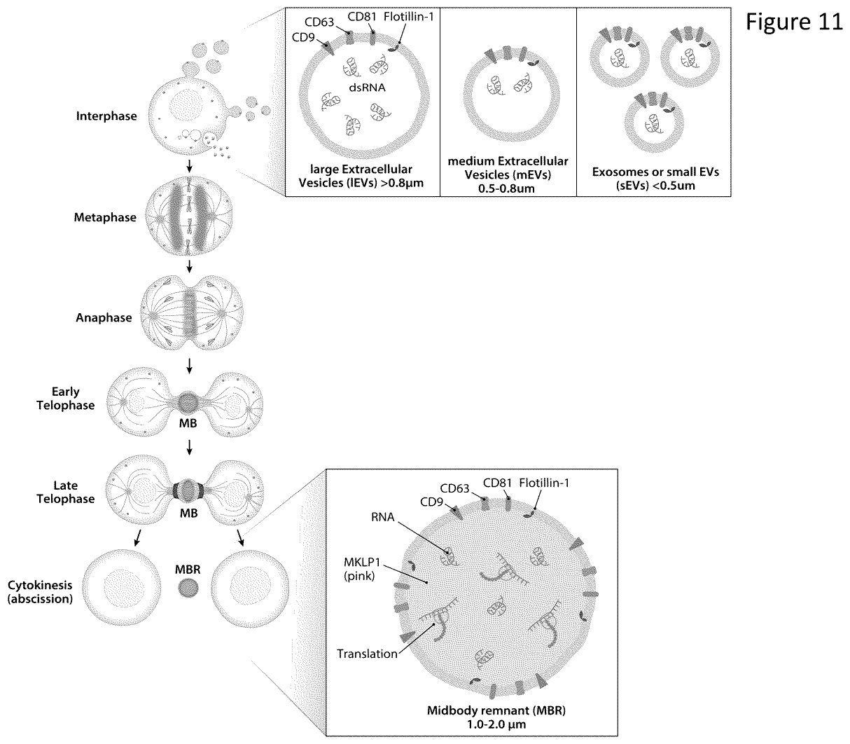

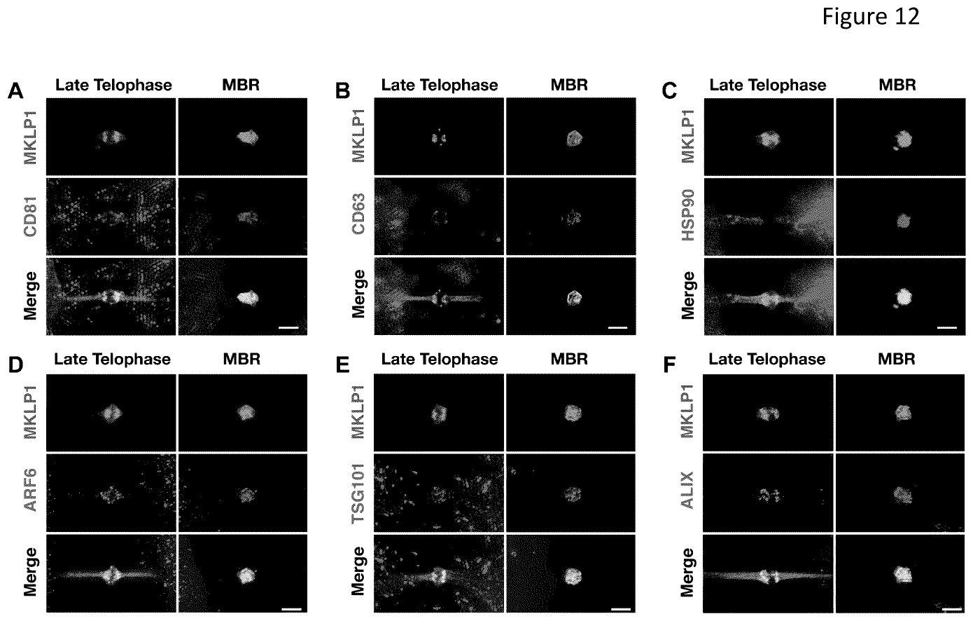

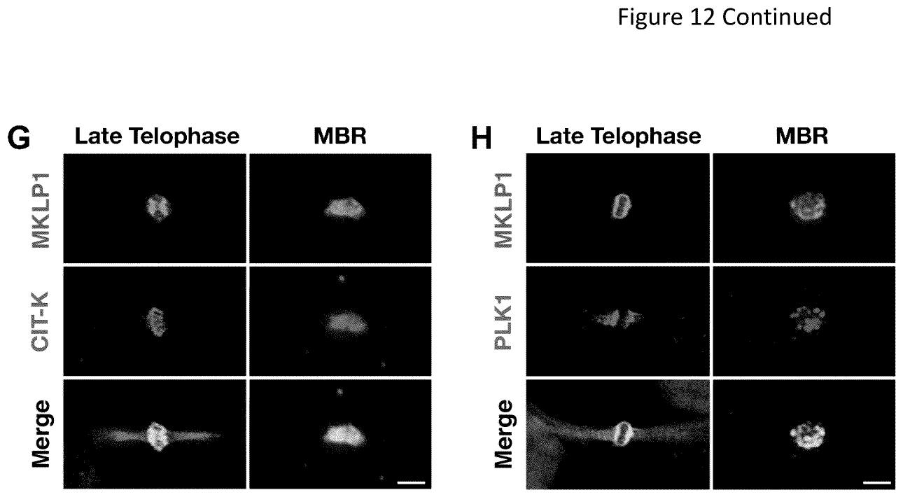

To determine if the biogenesis of large EVs occurs in mitosis, we have taken a multi-pronged approach, including imaging analysis, common and commercial EV isolation comparisons, cell cycle and cytokinesis blocks and translation activity measurements. We used image analysis to determine where canonical EV markers localize during mitosis with relation to MKLP1 in HeLa cells (FIG. 5 and FIG. 12), and where they localize on MBRs (FIG. 6). Next, we determined if common EV isolation methods, and the new methods we developed, isolate MBRs as well as other sizes of EVs (FIGS. 7 and 8). We blocked mitosis genetically with siRNA or chemically through drug treatment (RO-3306) and, as a result, we discovered a significant decrease in all sizes of EVs (FIG. 9). We then determined that in all isolated large EVs, only MBRs maintained translation activity (FIG. 10). Finally, we addressed the major ways EVs are generated from cells given our results and proposed a new model for the biogenesis of EVs (FIG. 11). The implications of this work are promising, because EVs serve as excellent vehicles for therapeutics and as factors for cancer and neurodegenerative disease diagnostics. We also suggest that the knowledge uncovered in this study provides the field a starting point to harness the power of the MBR, which is assembled during mitosis from the spindle midzone and midbody and is comprised of MKLP1, microtubules, RNA, and protein (Peterman 2019, Addi 2020, Park 2023, Farmer 2023, Skop 2004).

### Results

### EV Markers Assemble on Mitotic Structures and the Midbody During Mitosis.

To investigate the biogenesis of large EVs, we labeled HeLa (CCL2) cells with the canonical EV markers, CD9, CD81, flotillin-1, and dsRNA (Carnino 2019, Kowal 2017, Andreu 2014, Doyle 2019, Zaborowski 2015, Lischnig 2022, Cai 2021, Cai 2018, Rada 2022, Yoon 2020, O'Brien 2020, Yoshioka 2013), and visualized their localization patterns through the cell cycle using MKLP1 as a marker of the spindle midzone, midbody, and MBR (FIG. 5) (Mishima et al., 2004; Zhu et al., 2005; Hu et al., 2012).

CD9, CD81, flotillin-1, and dsRNA formed distinct puncta in the cell during interphase and throughout the cell cycle (FIG. 5A-D), but the puncta coalesced and concentrated at distinct sites during mitosis. CD9, CD81 and flotillin-1 demonstrated a similar mitotic pattern and are described collectively below (FIG. 5A-C). Briefly, EV markers formed a cloud of puncta around the metaphase plate with additional puncta dispersed throughout the cells during metaphase. At anaphase, EV markers decorated the spindle midzone and began to colocalize with MKLP1, a marker for spindle midzone microtubules. EV markers and MKLP1 formed a ring-like structure during early telophase, which is when the midbody structure begins to form as the cell membrane starts to pinch on both ends. In late telophase, EV markers and MKLP1 concentrated in the midbody structure. EV markers and MKLP1 remained evident in the MBR post abscission, which is after the MBR is jettisoned into the media or extracellular space. The distribution of dsRNA did not co-localize with MKLP1 throughout all the stages of mitosis but, as dsRNA is present throughout the cell and not just on the membrane like the other three markers this was to be expected. Here, dsRNA puncta were found just adjacent to the metaphase plate, at the spindle midzone, midbody and MBR (FIG. 5D).

Next, we evaluated other EV and midbody marker patterns during midbody and MBR stages. EV marker proteins CD63, CD81 (Campos-Silva 2019, Kowal 2017, Andreu 2014), HSP90, ALIX, TSG101 (Cordonnier 2017, Jeppesen 2023, Gurung 2021), ARF6 (Ghossoub et al., 2014), PLK1 (Ikawa et al., 2014), and CIT-K (Loomis et al., 2006; Bassi et al., 2013; Hessvik and Llorente, 2018) were used to label HeLa (CCL2) cells and imaged in late telophase and MBR stages (FIG. 12A-H). All of these markers were localized in the midbody region with MKLP1 in late telophase and MBR stages.

To determine the localization pattern of tetraspanins with MKLP1 in isolated HeLa MBRs, we used the Skop laboratory 1.5% PEG6000 isolation method and performed immunofluorescent staining using structured illumination microscopy (SIM). In the representative maximum Z projection images, CD9, CD63 and CD81, localized with the MBR marker MKLP1 either everywhere (CD9; 2A) or surrounding the MKLP1 (CD63 & CD81; FIG. 6B-C). The MBR line scan graph indicated that CD9 was dispersed throughout the whole MBR (FIG. 6D) whereas CD63 and CD81 were more abundant on the surface (FIGS. 6E and 6F). These results suggest that the canonical EV markers all co-localized with MKLP1, the midbody marker. This data suggests that the MBR is a large EV and that large EVs likely have their biogenesis in mitosis.

### Newly Developed EV Isolation Methods are Ideal for Isolating Large EVs or MBRs.

There are several common and commercial isolation methods used to isolate EVs, including ultracentrifugation followed by sucrose gradient (Suwakulsiri 2023, Ji 2020, Zhang 2023, Rai 2021), total exosome isolation reagent (TEIR) (Helwa et al., 2017; Martins et al., 2018; Skottvoll et al., 2018; Patel et al., 2019; Dash et al., 2021), immune capture by nanoparticles (Dynabeads) (Clayton et al., 2001; Oksvold et al., 2014; Fang et al., 2018; Lim et al., 2019), and 8% PEG6000 (Polyethylene Glycol) treatment (Rider et al., 2016). These methods have been used extensively to isolate all sizes of EVs; however, they are expensive and often lengthy protocols, and they are not specific enough to isolate only large or small EVs. In addition, many of them require the use of CD9 or CD81 as markers. Here, we describe two new methods of large EV/MBR isolation and compare them to canonical methods (FIG. 7).

We discovered that EV/MBR isolation using 7.5% PEG6000 treatment (Rider 2016), led to distorted and misshapen vesicles (Park et al., 2023b). Given this result, we sought to optimize isolation using a modified 8% PEG6000 isolation method derived from Rider et al., 2016 (Rider et al., 2016). We found that 1.5% PEG6000 maintained the membrane vesicle structure and size of large EVs or MBRs. The reduced PEG percentage did not interfere with the membrane fluidity of isolated EV/MBRs, and the shape was maintained during the high-speed centrifugation necessary for isolation (Park et al., 2023b).

To further optimize the specific isolation of large EVs or MBRs, we adapted two recently published EV isolation methods (Rider et al., 2016; Guru et al., 2022) with the goal of separating large EVs from small exosomes using size exclusion and precipitation. We combined and modified the methods: one using nonconjugated 20 nm PEG coated iron oxide nanoparticles and the other using 20 nm PEGylated gold nanoparticles conjugated to exosomal surface protein antibodies. PEGylated iron oxide nanoparticles trap unwanted proteins and tiny impurities in the reticular structures of PEG, which removes impurities using a permanent magnet and leaves behind pure exosomes in solution (Chang et al., 2018). The use of the gold particles adds weight to the vesicles, allowing them to be centrifuged at low speeds. Activation of the —COOH functional group on PEG, and conjugation to exosomal surface protein antibodies, like CD63, allows the gold particles to attach to the exosomes (Guru et al., 2022). We opted to use 30 nm PEGylated gold nanoparticles that were not conjugated to exosomal markers (Chang et al., 2018). We found that a 0.02% v/v concentration of 30 nm PEGylated gold particles in clear conditioned media was ideal for large EV/MBR isolation (Park et al., 2023b).

To compare our newly developed methods with other published and commercial methods, we isolated EVs according to each method and settled them onto coverslips for imaging using Poly-L-Lysine. To keep isolations comparable, cell culture media was harvested and centrifuged to remove cell debris before isolations were performed. Isolated EVs were fixed and labeled with anti-CD9 as a canonical EV marker and anti-MKLP1 as an MBR marker followed by confocal imaging. To avoid bias, confocal images were analyzed using a custom ImageJ macro developed in-house (sec Methods). The macro counted particles that were larger than 1 um in size in the CD9, MKLP1, and overlapping channels. The particles labeled with MKLP1 were classified as MBRs, and the particles labeled with only CD9 were classified as EVs. All the methods tested revealed that ˜96-98% of the isolated MBRs were also CD9 positive (FIG. 8A-B). The Skop Laboratory 1.5% PEG6000 (Skop PEG) and Skop Laboratory 30 nm gold particle (Skop gold) methods isolated the highest percentages of MBR double stained for CD9 and MKLP1 (98-99%) (Park et al., 2023b), and there was a significant difference between ultracentrifuge (UC) and TEIR (p=0.018), UC and Dynabeads (p=0.006), and UC and Skop PEG0 (p=0.025) (FIG. 8B). The fraction of MBRs among all isolated large EVs by each method was also calculated (FIG. 8C), revealing that all common and new methods isolate both large and small EVs. Although statistically insignificant, we found that the Skop gold method isolated the highest percentage of MBRs at approximately 65% of all EVs. TEIR and Dynabeads methods followed at 58% and 56%, respectively. Finally, the Skop PEG and ultracentrifuge methods had an MBR enrichment rate of 45% (FIG. 8C).

Cell Cycle Synchronization and siRNA Induced Cytokinetic Failures Leads to a Decrease in Total EV Production and a Significant Decrease in Large EVs

To determine the origins of all sizes of EVs, we used siRNA to silence either the expression of MKLP1, which inhibits cytokinesis (Zhu 2005, Janisch 2017, Makyio 2012, Durcan 2008) or RO-3306, a G2/M synchronization drug, which is known to inhibit proliferation (Vassilev et al., 2006) (FIG. 9A). To facilitate the process of quantification, the EVs labeled with either CD9 or CD63 were separated into classes based on their diameter (FIG. 9B). MBRs were classified as large EVs positive for both CD9 (or CD63) and MKLP1 with a diameter of 0.8 μm or larger. Large EVs (lEVs) had a diameter larger than 0.8 μm, medium EVs (mEVs) had a diameter of 0.5 μm to 0.8 μm and small EVs (sEVs) had a diameter of 0.5 μm or lower.

Next, we analyzed EVs released from HeLa cells treated with either control or MKLP1 siRNAs. Cells treated with MKLP1 siRNA for 48 hours suffered cytokinetic failure, and 90% of the cells showed multiple nuclei or bi-nucleation (FIG. 9C). Next, we isolated EVs using the Skop Lab 1.5% PEG6000 method and quantified the number of different classes of EVs released. Compared to the control group, the number of EVs released decreased significantly in all four classes (FIG. 9D-9F), even small EVs (FIG. 9F). The total number of lEVs and MBRs released from cells treated with MKLP1 siRNA decreased by 68.84% and 68.01% respectively (FIG. 9D). The total number of mEVs released decreased by 76.69% (FIG. 9E), and the total number of sEVs released decreased by 34.91%. (FIG. 9F). We were surprised to see a significant decrease in all size classes after blocking cytokinesis given the literature, especially with regard to the mEVs (0.5 um-0.8 um), which decreased by over 76%. This data suggests that mitosis generates EVs of all sizes, which might not be surprising as cancer cells tend to shed way more EVs than other cells (Rai 2021, Xu 2018, Suwakulsiri 2023, Karbanová 2024, Zocco 2014, Schmidtmann 2023, Kilinc 2021, Ferguson 2022, Suwakulsiri 2024).

To investigate EV release during a cell cycle block, we treated HeLa cells with the CDK1 inhibitor RO-3306 or DMSO control for 24 hours. The EVs were then harvested using Skop Lab 1.5% PEG6000 method similarly as to the MKLP1 siRNA treatment in FIG. 9A. Cells treated with RO-3306, were primarily halted in G2 or in metaphase as judged by DAPI staining (FIG. 9G). Compared to the control group, the total numbers of EV released decreased in all four classes. The total number of lEVs and MBRs released from cells treated with RO-3306 decreased by 62.34% and 62.22% respectively (FIG. 9H). The total number of mEVs released decreased by 33.35% (FIG. 9I) and the total number of sEV released decreased by 47.74% (FIG. 9J). The cell cycle blocks affected the biogenesis of the large EVs more so than the mEVs or sEVs, which indicated that the biogenesis of large EV may primarily come from mitotic events.

Overall, these two experiments suggest that preventing cytokinesis or blocking the cell cycle affects the biogenesis of all sizes of EVs, but primarily affect the biogenesis of the large EVs (62-68% of large EVs and MBRs are lost after these treatments). The MKLP1 siRNA treatment suggests that both large EVs and medium-sized EVs are primarily generated in mitosis as there is a 68%-76% loss of medium to large EVs/MBRs loss with this treatment. The small EVs appear to be generated from both interphase and mitotic cells, with interphase generating about ˜59% and mitosis generating about ˜41% of the small EVs.

### EV Isolation Kits Reveal That Large EVs Have the Capacity to Translate.

As our lab recently discovered that the midbody and MBR are sites of active translation (Park et al., 2023a), we sought to determine if common and commercial methods of isolating EVs also preserve translation capacity. Puromycin antibody labeling was used as a proxy for active translation activity because the localization pattern is similar to HPG-Click-IT activity (Park et al., 2023a). Here, cleared culture media was treated with puromycin for 10 minutes at 37° C. If large EVs in the media were translating, translation would stop after incorporation of puromycin into the growing polypeptide chain (Aviner, 2020; Enam et al., 2020). The media was then subjected to each isolation method and the EV mixture was centrifuged onto cover glasses and labeled with anti-MKLP1 and anti-puromycin (FIG. 10A). Imaging revealed all isolated MBRs were labeled with MKLP1 and puromycin, showing an overlap between the two channels (FIG. 10B, dashed boxes with puromycin (Cyan) and MKLP1 (Magenta). Particles in the puromycin and MKLP1 channels were counted using the same custom ImageJ macro (see Methods). Analysis showed that the Dynabead method had the highest percentage of MBRs with a puromycin tag at 99.6% followed closely by Skop Lab 1.5% PEG6000 method at 99% (FIG. 10C). The ultracentrifugation method was at 98%, and the TEIR and Skop Lab 30 nm gold particle isolation methods at 96% and 95% respectively. Of note, the differences between these results were not statistically significant. Additionally, particles were detected in the puromycin channel that did not express MKLP1 (FIG. 10B, dotted boxes with CYAN only), suggesting that all EV isolation methods isolated additional particles with translation capacity. These nanometer particles could be remains of the HeLa cell cytoplasm or other exosomes. The high centrifugal force necessary to clear culture media can also damage the cell membrane, and the cytoplasm can ‘leak’ into the media. These experiments did not prove that large EVs or MBRs in media maintain translation after isolation, because puromycin treatment was performed before EV isolation. To answer this question, EV isolation was also performed prior to puromycin treatment. After incubation and isolation, EV mixtures were treated with puromycin for 10 minutes at 37° C. The resulting EVs were labeled with anti-puromycin and anti-MKLP1 (FIG. 10D). We found that MBRs maintained the ability to translate after isolation. Indeed, all MBRs isolated using common, commercial, and our new isolation methods were puromycin positive, with no statistically significant difference between them, indicating that isolated MBRs retain the capacity to translate regardless of the isolation method.

### Discussion

The central aim of this study was to elucidate the biogenesis of large extracellular vesicles (EVs) in the size range of 1000-2000 nm, which has remained enigmatic despite extensive research in the EV field. We hypothesized that these large EVs originate primarily from mitosis, contrary to the prevailing assumption that all EVs arise from interphase cells. First, we determined when and where the common EV markers, CD9, CD81, Flotillin and dsRNA, localize during mitosis and post-mitotically within the midbody remnant. Next, we used common, commercial and newly developed methods to isolate EVs and determined if these EVs also label with MKLP1, the midbody marker. We then blocked mitosis using MKLP1 siRNA and a small molecule to block the cell cycle, to determine what size EVs were affected by these treatments. Lastly, we determined if these common, commercial and newly developed isolation methods also preserve translation activity of the MBR class.

The MBR has been more recently appreciated over the last several years, but has a long history going back to 1891 with the discovery by Walther Flemming (Addi 2020, Mullins 1979,D'Avino 2017, Mullins 1982, Matuliene 2002, Pinheiro 2014, Otegui 2005, Dionne 2015, Antanavičiūtė 2018, Farmer 2022, Chen 2012, Peterman 2019, Peterman 2019, Skop 2004). Work over the last decade has revealed that the MBR is not only generated during abscission, but jettisoned into the media of cultured cells and often internalized by adjacent cells (Crowell 2014, Crowell 2013, Peterman 2019,Addi 2020). Equally important are the active translation properties of the MBR that our lab and others have identified (Farmer et al., 2023; Jung et al., 2023; Park et al., 2023a). Given this, the MBR has been positioned as a potential mediator of cell fate (Addi 2020, Crowell 2014, Crowell 2013, Peterman 2019, Chaigne 2020, Park 2023, Farmer 2022), particularly given the unique RNA and protein cargo that is assembled within the structure during mitosis. Despite all of this work, this unique signalling organelle has remained unappreciated until recent genomic and proteomic work in the EV field revealed cytokinesis markers for large EVs (Suwakulsiri 2023, Nikonorova 2022, Kowal 2016, Martinez-Greene 2021, Li 2023, Rodosthenous 2020, Berardocco 2017, Luo 2022, Lischnig 2022). It became clear that the MBR is an extracellular vesicle given the presence of KIF23/MKLP1, the master gene for cytokinesis, in many of the large EV datasets (Lischnig 2022, Suwakulsiri 2023). However, many questions remain as to the exact origin of large EVs given recent findings.

One of these questions has been the biogenesis or origin of large EVs, which are thought to be released through exocytosis from cells primarily in interphase (as judged by numerous sketched models found in reviews) (Gurunathan 2021, Colombo 2014, Gurunathan 2021, Liegertová 2023, Niel 2022, Gurung 2021, Niel 2018, Akers 2013). Although alternative biogenesis pathways have been suggested by one EV group, this lab proposed perhaps the MBR was a new large EV group (Gurunathan 2021, Colombo 2014, Gurunathan 2021, Liegertová 2023, Niel 2022, Gurung 2021, Niel 2018, Akers 2013). However, Westerns of density-separated large EV classes, including isolated microparticles (MPs) and MBRs, revealed that vesicles of different densities both contain KIF23/MKLP1, suggesting that these two classes originate in mitosis and/or belong to the exact same class of large EVs. Lastly, we know that EVs are then released into the cell culture media and can communicate with adjacent cells (Tetta et al., 2013; Pitt et al., 2016; Tkach and Théry, 2016; Maacha et al., 2019), similar to what has been observed for MBRs (Crowell 2014, Peterman 2019, Peterman 2019, Chaigne 2020, Addi 2020). From these findings, we hypothesized that MBRs are synonymous with large EVs and are generated by mitotic cells.

For over 14 years, the EV field has documented that cancer cells release more EVs than their non-cancerous counterparts (Sandim and Monteiro, 2020). Tumors contain multiple cell types in all different stages of the cell cycle, and 10-20% of tumors have been shown to contain mitotic cycling cells (Adams et al., 2016; Donovan et al., 2021; Urezkova et al., 2021; Ibrahim et al., 2022). These considerations coupled with the increased proliferation index of cancer cells, suggest that it is highly likely that MBRs are being generated and released at higher rates in cancer cells than normal cells. To investigate, we mapped the localization of common EV markers throughout the cell cycle to determine when and how EVs may be generated. Additionally, we showed that known midbody markers (ARF6, TSG101, ALIX, CIT-K, and PLK1) which have been used as EV markers or found in them (Clancy 2022, Yoshioka 2013, Colombo 2014, Suwakulsiri 2023, Loomis 2006) clearly co-localized with MKLP1 (FIG. 12). Based on our findings, we suggest a new model for EV biogenesis (FIG. 11). In this model, common EV markers are dispersed throughout the cell membrane in interphase, and small, medium and large EVs are released from the cell membrane. However, during the stages of mitosis, EV markers move in a highly localized and enriched manner to mitotic structures. They form a cloud around the metaphase plate in metaphase, localize to the spindle midzone in anaphase, and are found in the midbody and MBR in telophase and during abscission. MKLP1, which is a midbody and MBR marker, demonstrated the same localization pattern as EV markers starting in anaphase through MBR stage. MBRs released into the extracellular space showed a clear overlap between EV markers and MKLP1. Our findings highlight a mitotic pathway for the generation of large EVs showing that canonical EV markers assemble during mitosis in the spindle midzone, midbody and MBR.

To determine if common and commercial EV isolation methods isolate MBRs, we compared these methods by assessing known EV and MBR markers after isolation. Our reasoning for doing this was to assess the literature surrounding the activity and impact of EVs on cell biology. In addition, the interpretation of results found in the EV literature is broad and is inherently dependent on the isolation methods used. Over the years, Nanoparticle tracking analysis (NTA) and western blotting have been used to characterize isolated EVs; however, whilst NTA measures size it has an upper limit of 1000 nm therefore excluding most large EVs and MBRs, and Western blotting indicates protein content without differentiating between EV sizes, so neither method truly assesses both the relative size of particles and the proteins that colocalize with them. To improve the characterization of isolated particles, we used a quantitative imaging approach to measure CD9 or CD63, canonical EV markers (Termini 2017, Andreu 2014), and MKLP1, a marker of the midbody and MBR (Hu et al., 2012; Tkach and Théry, 2016; Raposo and Stahl, 2019). Automated counting was performed on the images obtained from EV isolation using a custom ImageJ macro that was developed in-house (see Methods). This macro counted the number of CD9 or CD63 positive particles, MKLP1 positive particles, and dual positive particles. The resulting counts were transformed into percentages of the total number of particles, which enabled comparisons between the methods without respect to the number of EVs isolated. Our results also showed that ˜97% of large EVs labeled with CD9 were also MKLP1 positive (FIG. 6), suggesting that the large EVs are indeed MBRs that originate in mitosis. This observation is consistent with a recent proteomic analysis of small and large EVs, which showed that small and large EVs were positive for almost all EV markers whereas only large EVs were positive for RACGAP1 and KIF23 (MKLP1) (Lischnig 2022). We did also identify a large EV class that only labeled with CD63 and not MKLP1, suggesting that there is a class of large EVs that do not have a mitotic origin. We are unable to discern where this class originates or whether it belongs to what some are calling microparticles (Suwakulsiri 2023, Rai 2021, Suwakulsiri 2024). Our study also suggests that CD9 or CD63, does not differentiate between large and small EVs but that MKLP1 was clearly identified as a specific marker for the MBRs, suggesting that MKLP1 can be used to identify large EVs from the rest of the EV population.

Probably our most significant experiments we performed were where we blocked cells in either cytokinesis or the cell cycle. Both treatments led to significant decreases in all sizes of EVs including large, medium and small extracellular vesicles (FIG. 9). We were surprised to find that these cell cycle and mitotic blocks also led to decreases in smaller vesicles, as the current understanding of exosome or small EV production occurs via exocytic release (Beshbishy 2020, Gurunathan 2021, Colombo 2014, Suwakulsiri 2023, Mardi 2023, Zhang 2018, McAndrews 2019, Latifkar 2019, Niel 2018, Andreu 2014, Mathieu 2019, Kalluri 2020, Gurung 2021, Akers 2013), but this could perhaps be due to the stress caused on cells using these treatments or drugs, which might block exocytosis (Markina-Iñarrairaegui et al., 2013). These treatments could alternatively lead cells to enter a senescent state, which could lead to less EV release, yet senescent cells often release more exosomes (Lehmann et al., 2008). What we do understand from these experiments, is that blocks in the cell cycle and mitosis lead to a significant decrease in the amount of MBRs (which is expected as mitosis does not occur), but also other large non-MKLP1-labeled EVs, whose origin is unclear. Equally, medium sized EVs show a significant decrease after MKLP1 siRNA treatment, suggesting that perhaps some medium-sized EVs originate in mitosis. The RO-3306 treatment which blocks the cell cycle in G2 phase through metaphase (Vassilev et al., 2006), suggest that all sizes of EVs can originate during this cell cycle window. While there is accumulating evidence that EV biogenesis occurs via exocytic pathways or membrane shedding (Gurunathan 2021, Liegertová 2023,Colombo 2014, Théry 2018, Latifkar 2019, Margolis 2019, Gurung 2021, Akers 2013), it is clear that mitosis is another pathway that generates significant amounts of EVs, knowledge particularly useful when using EVs for therapeutics and cancer diagnostics.

Lastly, we have shown that large EVs or MBRs labeled with puromycin were present in all EV isolation methods tested. This implies that all methods used in research to date have likely been contaminated with translating large EVs or MBRs. To note, there were even smaller particles identified in our studies that labeled with puromycin (FIG. 10B; dotted Cyan/PURO only box), perhaps from cellular debris depending on the isolation methods used or smaller MBRs. The translation activity is a unique function of this class of large EVs. Large EVs or MBRs also have a higher cargo capacity and can carry larger more specific payloads than smaller EVs or exosomes, including rRNA and mRNA which are enriched in large EVs (Gurunathan 2021, Liegertová 2023, Colombo 2014, Théry 2018, Latifkar 2019, Margolis 2019, Gurung 2021, Akers 2013). Overall, our data suggests that this unique class of EVs may have a greater biological impact given the large payload and also translation activity, especially during cell-cell communication, viral transmission, and their use as drug delivery vehicles.

In summary, we demonstrate that the mitotic origin of large EVs as MBRs, a finding that challenges existing paradigms and has broad implications for EV research, cancer diagnosis, and therapeutic engineering. Large translating EVs have a different mechanism of biogenesis that is distinct from membrane budding and exocytosis of microvesicles/microparticles and exosomes (Gurunathan 2021, Liegertová 2023, Colombo 2014, Théry 2018, Latifkar 2019, Margolis 2019, Gurung 2021, Akers 2013). These large translating EVs are also molecularly distinct from exosomes (Lischnig 2022, Park 2023), with a mitotic origin, and have MKLP1 as a unique marker. Cancer cells divide rapidly and therefore produce high quantities of MKLP1-labeled large EVs (Suwakulsiri 2023, Suwakulsiri 2024, Lischnig 2022, Park 2023, Rai 2021). This unique mitotic EVs class can therefore serve as diagnostic markers for cancer, as MKLP1-positive MBRs present in liquid biopsies from cancer patients could be counted and compared to normally cycling cells. Another implication of this work is the engineering potential for drug delivery and therapeutics for many diseases, including neurodegeneration and cancer, since cells can be genetically engineered to assemble therapeutic cargo in their MBRs during mitosis using MKLP1 as a specific target. We provide a novel perspective on EV biogenesis and identify unique markers for this class of large, translating EVs, paving the way for future investigations into their biological roles and clinical applications.

### Methods:

Cell culture: HcLa (ATCC® CCL-2™) cells (Douglas et al., 2010) were cultured in DMEM/high-glucose (Thermo Fisher Scientific, Cat #11965-118) or DMEM/F12 HEPES (Thermo Fisher Scientific, Cat #11-330-057), supplemented with 10% fetal bovine serum (FBS) and 1% penicillin/streptomycin (p/s), at 37° C. in a humidified incubator at 5% CO₂. Chemicals were used in cultures at the indicated concentrations: 91 μM puromycin (Sigma-Aldrich, Cat #P8833) and 9 μM RO-3306 (Santa Cruz, Cat #sc358700).

EV purification—TEIR: EVs were purified from culture media using a Total Exosome Isolation Reagent (TEIR, Thermo Fisher Scientific, Cat #4478359). Briefly, HeLa cells were grown in a T175 flask for one week and fed with DMEM/F12 media, supplemented with 10% FBS and 1% p/s, every 2 days. Culture media (10 ml) was collected and centrifuged at 1,000×g for 10 min to remove cell debris. TEIR was added at a volume equivalent to half the remaining volume of cleared culture media (i.e., 5 ml of TEIR solution would be added to 10 ml culture media). The mixture was incubated overnight at 4° C. and then centrifuged at 10,000×g for 1 hr at 4° C. The resulting pellet was dissolved in 1 ml PBS. The mixture was placed on a poly-L-lysine-coated 18 mm #1.5 thickness circular cover glass and incubated overnight to allow EVs to settle onto the coverslip. EVs were fixed in 3.7% PFA with 0.3% Triton X-100 in PHEM buffer at RT for 15 min.

Dynabeads: EVs isolated using TEIR were purified with Protein G Dynabeads (Thermo Fisher Scientific, Cat #10003D). Dynabeads were vortexed, and 1.5 mg (50 μl) of Dynabeads were placed in an Eppendorf tube on a DynaMag (Thermo Fisher Scientific, Cat #12321D). The liquid was aspirated from the Dynabeads, and 10 μg of CD81 antibody (Santa Cruz, Cat #SC-23952) diluted in 200 μl PBS with Tween 20 was added to the Dynabeads. The Dynabeads were incubated for 40 min at 37° C. on a rocker to allow Dynabead-antibody conjugation. The beads were placed on the DynaMag again, and the liquid was aspirated, followed by washing with 200 μl PBS to remove excess antibodies. Conjugated dynabeads were incubated with 1ml of TEIR-derived EV solution overnight at 4° C. on a rocker. After incubation, the tube was placed on the DynaMag, and the excess liquid was aspirated. To elute EVs from the Dynabeads, 100 μl of 1× Nupage sample reducing agent (Thermo Fisher Scientific, Cat #NP0004) diluted in PBS was added to the beads and incubated at 37° C. for 1 hr on a rocker. After isolating the liquid from the Dynabeads using the DynaMag, the solution was placed onto a poly-L-lysine-coated 18 mm #1.5 thickness circular cover glass and incubated at 4° C. overnight to allow EVs to settle. EVs were fixed in 3.7% PFA with 0.3% Triton X-100 in PHEM buffer at RT for 15 min.

Ultracentrifugation: EVs/MBRs were isolated by ultracentrifugation of cleared culture media adapted from Peterman, et al., 2019. Hela cells were grown in four T175 flasks with 20 ml DMEM/F12 media, supplemented with 10% FBS and 1% p/s, for 1 week. Media was changed on day 5, and cells were grown until 80% confluent. Cultured media was collected and centrifuged at 1,000×g for 10 min to clear cell debris. Cleared cultured media was transferred to ultracentrifuge tubes (Thermofisher, Cat #3118-0050) and centrifuged at 10,000×g for 30 min at 4° C. After aspiration, the pellets were combined and dissolved in 1 ml sterile PBS. The solution was transferred to 1.5 ml microcentrifuge tubes and centrifuged at 10,000×g for 30 min at 4° C. After aspiration, the pellet was resuspended in nuclease-free water with 6 mM MgCl₂, 250 mM sucrose, and 250 mM NaCl to a final volume of 200 μl. A density gradient was prepared in a 1.5 ml tube by layering 300 μl of 40% glycerol followed by 400 μl each of 1M and 2M sucrose solutions consisting of 20 mM HEPES, 6 mM MgCl₂, 1 mM dithiothreitol, and 100 mM NaCl at pH 7.4. The resuspended pellet was layered onto the density gradient and centrifuged at 3,000×g for 20 min at 4° C. Immediately after centrifugation, the band between the 2M and 1M sucrose solutions was collected and centrifuged at 10,000×g for 30 mins at 4° C. After aspiration, the invisible pellet was dissolved in 40 μl sterile PBS. The dissolved MBRs were placed on poly-L-lysine-coated 18 mm #1.5 circular cover glass and incubated overnight at 4° C. to allow MBRs to settle onto the cover glass. MBRs were fixed in 3.7% PFA with 0.3% Triton X-100 in PHEM buffer at RT for 15 min.

Skop Laboratory 1.5% PEG6000 method (Park et al., 2023b): HeLa cells were grown in a T175 flask for one week and fed with DMEM/F12 media, supplemented with 10% FBS and 1% p/s, every 2 days. Culture media (10 ml) was collected and centrifuged at 1,000×g for 10 min to remove cell debris. Cleared culture media (9.5 ml) was transferred to a 15 ml conical tube (Falcon, Cat #352097), and 500 μl of 30% polyethylene glycol 6000 (PEG, Sigma-Aldrich, Cat #81253-250G) in DPBS (Thermo Scientific, Cat #45000-430) was added to a final concentration of 1.5% (v/v). The mixture was vortexed and incubated overnight at 4° C. The mixture was sedimented onto poly-L-lysine-coated 18 mm #1 circular cover glass by centrifugation at 3,000×g for 10 min. EVs/MBRs were fixed in 3.7% PFA with 0.3% Triton X-100 in PHEM buffer at RT for 15 min.

Skop Laboratory 30 nm gold nanoparticles method (Park et al., 2023b): HeLa cells were grown in a T175 flask for one week and fed with DMEM/F12 media, supplemented with 10% FBS and 1% p/s, every 2 days. Culture media (10 ml) was collected and centrifuged at 1,000×g for 10 min to remove cell debris. Cleared culture media was transferred to a 15 ml conical tube, and 2 μl of 30 nm gold nanoparticles coated with PEG5000 (Sigma-Aldrich, Cat #765732-1ML) were added. The mixture was vortexed and incubated overnight at 4° C. The mixture was sedimented on poly-L-lysine-coated 18 mm #1.5 circular cover glass by centrifugation at 3,000×g for 10 min. EVs/MBRs were fixed in 3.7% PFA with 0.3% Triton X-100 in PHEM buffer at RT for 15 min.

siRNA experiments: HeLa cells were grown for 24 hr in 150 mm culture dishes with a 18 mm #1.5 thickness circular cover glass in DMEM/high-glucose supplemented with 10% FBS and 1% p/s to 50% confluence. After 24 hr, 120 μl siRNA (final concentration 80 pmols) (Santa Cruz) and 90 μl lipofectamine RNAiMAX (Invitrogen) were added to 1.5 ml Opti-MEM media (Thermo Fisher, Cat #31985062), mixed, incubated for 5 min at room temperature. and added to the relevant cells and incubated for 5 hr. The media was replaced to reduce siRNA toxicity and the transfected cells cultured for 48 hr before EV isolation using the Skop lab 1.5% PEG6000 method. The cells on the cover glass were fixed in 3.7% PFA with 0.3% Triton X-100 in PHEM buffer at RT for 15 min.

RO-3306 inhibitor treatment: HeLa cells were grown for 24 hr in 150 mm culture dishes with a 18 mm #1.5 thickness circular cover glass in DMEM/high glucose supplemented with 10% FBS and 1% p/s to 60% confluence. 9 μM of RO-3306 or the equivalent volume of DMSO was added to the cells in 20 mL DMEM high glucose with 10% FBS and 1% p/s and incubated for 24 hours. EV were isolated from the media using the Skop Lab 1.5% PEG6000 method, and the cells on the cover glass were fixed in 3.7% PFA with 0.3% Triton X-100 in PHEM buffer at RT for 15 min.

Visualizing active protein translation using puromycin labeling: To visualize active translation, cell culture supernatant and isolated MBRs on cover glass were pulsed with 91 μM puromycin (Millipore Sigma, Cat #P8833) in culture media for 10 min at 37° C., fixed as described, and immunostained using anti-puromycin antibodies (Millipore Sigma, Cat #MABE343). Samples were co-incubated with anti-MKLP1 antibodies (Novus Biologicals, Cat #NBP2-56923) as a marker for MBRs.

Immunofluorescence: HeLa cells were used for immunofluorescence assays of midbodies or MBRs, respectively. Cells were cultured on 18 mm circular cover glasses and fixed in 3.7% PFA with 0.3% Triton X-100 in PHEM buffer at RT for 10 min. Non-specific antibody binding was blocked by 1 hr incubation in PHEM buffer containing 3% BSA (Sigma-Aldrich, Cat #A2153) (blocking buffer) at RT, followed by incubation with the relevant primary antibodies in blocking buffer at 4° C. overnight. Cover glasses were washed three times 5 min with PBST (PBS, 0.1% Tween 20), incubated with relevant secondary antibody in blocking buffer for 2 hr at RT then washed 3 times 5 min with PBST. Cover glasses were mounted on slides using either VectaShield with DAPI (Fisher Scientific, Cat #nc1601055) or Fluoro-Gel mounting medium (Electron Microscopy Sciences, Cat #17985-30) on Colorfast Microscope Slides (Fisherbrand, Cat #12-550-413). Slides were analyzed using structured illumination microscopy (SIM), confocal microscopy or Revolve scope.

N-SIM imaging: Fluorescence images were collected on a motorized inverted Eclipse Ti-E structured illumination microscope (Nikon). Images were captured on the Andor iXon 897 EMCCD camera (Andor Technology). ImageJ was used to generate maximum intensity projections.

Confocal imaging: Fluorescence images were collected using a Nikon AIR-Si+ confocal microscope. Multichannel images were captured by high-sensitivity GaAsP detectors using 561 nm and 640 nm lasers and a 60× objective. ImageJ was used to generate maximum intensity projections.

Revolve scope imaging: Fluorescence images were captured by a 5MP CMOS color camera on a RVL2-K microscope by ECHO (revolve microscope).

Particle counting: Maximum Z-Projection images of isolated EVs/MBRs stained with MKLP1 and other EV markers were used in these analyses. EV/MBR counting was performed using ImageJ, and a custom macro tool (https://github.com/arskop/midbody.git) was written to automate image analysis using a series of image analysis techniques available in the Fiji software package. Multichannel images were imported and subjected to maximum Z projection, followed by application of an auto threshold using the Moments function. Subsequently, the Analyze Particle function was employed to count particles larger than the predefined size threshold of 0.8 (0.8 μm in diameter) and with a circularity value greater than 0.1. To identify overlapping particles, images were split into two channels (488 and 647 nm channels) and combined using the Image Calculator ‘AND’ function, and the Analyze Particles function was applied with parameters identical to the preceding step. Particle counts were subsequently exported to separate tables to facilitate data processing in Microsoft Excel and Prism.

Differentially Sized Particle Counting: MBR, lEV, mEV, and sEV counting was performed using a custom automated ImageJ macro tool (github.com/arskop/midbody.git) based on the diameter of particles. Images were imported into imageJ, automatic thresholding with “Moments” method was applied to each image to distinguish the features of interest. To identify MBRs, particles positive for MKLP1 and CD9 or CD63 markers were identified using AND function using Image Calculator. Overlapping particles with both markers larger than 0.8 μm diameter were counted as MBRs. Counts for EVs of different sizes were acquired using Analyze Particle function with pre-defined sizes: sEV (<0.5 μm), mEV (0.5-0.8 μm), and lEV (>0.8 μm). Particles counts were exported into Microsoft Excel and Prism for analysis.

Statistical analysis: All graphs were drawn on on GraphPad Prism Software (Graphpad, San Diego, CA). Quantitative data represented as mean±standard error of mean (s.e.m). Statistical analysis was performed using R software (Wilcoxon Rank Sum Test) with P value <0.05 considered as statistically significant (*p<0.05, **p<0.01, ***p<0.001, ****p<0.0001). For all immunofluorescence HeLa cell imaging analysis, at least 13 sample images were analyzed to report localization pattern. For MBR isolation experiments, isolation was done in triplicate with at least 5 different imaging fields imaged for each cover glass, and at least 15 images were analyzed for each condition.

### REFERENCES

Adams, DL, Adams, DK, Stefansson, S, Haudenschild, C, Martin, SS, Charpentier, M, Chumsri, S, Cristofanilli, M, Tang, C-M, and Alpaugh, RK (2016). Mitosis in circulating tumor cells stratifies highly aggressive breast carcinomas. Breast Cancer Research 18, 44.
Addi, C, Presle, A, Frémont, S, Cuvelier, F, Rocancourt, M, Milin, F, Schmutz, S, Chamot-Rooke, J, Douché, T, Duchateau, M, et al. (2020). The Flemmingsome reveals an ESCRT-to-membrane coupling via ALIX/syntenin/syndecan-4 required for completion of cytokinesis. Nature Communications 11, 1941.
Akers, JC, Gonda, D, Kim, R, Carter, BS, and Chen, CC (2013). Biogenesis of extracellular vesicles (EV): exosomes, microvesicles, retrovirus-like vesicles, and apoptotic bodies. J Neuro-Oncol 113, 1-11.
Andreu, Z, and Yáñez-Mó, M (2014). Tetraspanins in Extracellular Vesicle Formation and Function. Front Immunol 5, 442.
Antanavičiūtė, I, Gibieža, P, Prekeris, R, and Skeberdis, V (2018). Midbody: From the Regulator of Cytokinesis to Postmitotic Signaling Organelle. Medicina 54, 53.
Aviner, R (2020). The science of puromycin: From studies of ribosome function to applications in biotechnology. Computational and Structural Biotechnology Journal 18, 1074-1083.
Bassi, ZI, Audusseau, M, Riparbelli, MG, Callaini, G, and D'Avino, PP (2013). Citron kinase controls a molecular network required for midbody formation in cytokinesis. Proceedings of the National Academy of Sciences of the United States of America 110, 9782-9787.
Berardocco, M, Radeghieri, A, Busatto, S, Gallorini, M, Raggi, C, Gissi, C, D'Agnano, I, Bergese, P, Felsani, A, and Berardi, AC (2017). RNA-seq reveals distinctive RNA profiles of small extracellular vesicles from different human liver cancer cell lines. Oncotarget 8, 82920-82939.
Beshbishy, AM, Alghamdi, S, Onyiche, TE, Zahoor, M, Rivero-Perez, N, Zaragoza-Bastida, A, Ghorab, MA, Meshaal, AK, El-Esawi, MA, Hetta, HF, et al. (2020). Biogenesis, Biologic Function and Clinical Potential of Exosomes in Different Diseases. Applied Sciences 10, 4428.
Brennan, K, Martin, K, FitzGerald, SP, O'Sullivan, J, Wu, Y, Blanco, A, Richardson, C, and Gee, MMM (2020). A comparison of methods for the isolation and separation of extracellular vesicles from protein and lipid particles in human serum. Scientific Reports 10, 1039.
Cai, Q, He, B, Wang, S, Fletcher, S, Niu, D, Mitter, N, Birch, PRJ, and Jin, H (2021). Message in a Bubble: Shuttling Small RNAs and Proteins Between Cells and Interacting Organisms Using Extracellular Vesicles. Annual Review of Plant Biology 72, 497-524.
Cai, Q, Qiao, L, Wang, M, He, B, Lin, F-M, Palmquist, J, Huang, H-D, and Jin, H (2018). Plants send small RNAs in extracellular vesicles to fungal pathogen to silence virulence genes. Science 360, eaar4142.
Campos-Silva, C, Suárez, H, Jara-Acevedo, R, Linares-Espinós, E, Martinez-Piñeiro, L, Yáñez-Mó, M, and Valés-Gómez, M (2019). High sensitivity detection of extracellular vesicles immune-captured from urine by conventional flow cytometry. Scientific Reports 9, 2042.
Carnino, JM, Lee, H, and Jin, Y (2019). Isolation and characterization of extracellular vesicles from Broncho-alveolar lavage fluid: a review and comparison of different methods. Respiratory Research 20, 240.
Chaigne, A, and Brunet, T (2022). Incomplete abscission and cytoplasmic bridges in the evolution of eukaryotic multicellularity. Current Biology 32, R385-R397.
Chaigne, A, Labouesse, C, White, IJ, Agnew, M, Hannezo, E, Chalut, KJ, and Paluch, EK (2020). Abscission Couples Cell Division to Embryonic Stem Cell Fate. Developmental Cell 55, 195-208.e5.
Chang, M, Chang, Y-J, Chao, PY, and Yu, Q (2018). Exosome purification based on PEG-coated Fe3O4 nanoparticles. PLOS ONE 13, e0199438.
Chen, C-T, Ettinger, AW, Huttner, WB, and Doxsey, SJ (2012). Resurrecting remnants: the lives of post-mitotic midbodies. Trends Cell Biol 23, 118-128.
Chen, M, Xu, R, Ji, H, Greening, DW, Rai, A, Izumikawa, K, Ishikawa, H, Takahashi, N, and Simpson, RJ (2016). Transcriptome and long noncoding RNA sequencing of three extracellular vesicle subtypes released from the human colon cancer LIM1863 cell line. Scientific Reports 6, 38397.
Cheng, P, Wang, X, Liu, Q, Yang, T, Qu, H, and Zhou, H (2023). Extracellular vesicles mediate biological information delivery: A double-edged sword in cardiac remodeling after myocardial infarction. Frontiers in Pharmacology 14, 1067992.
Clancy, JW, Sheehan, CS, Boomgarden, AC, and D'Souza-Schorey, C (2022). Recruitment of DNA to tumor-derived microvesicles. Cell Rep 38, 110443.
Clayton, A, Court, J, Navabi, H, Adams, M, Mason, MD, Hobot, JA, Newman, GR, and Jasani, B (2001). Analysis of antigen presenting cell derived exosomes, based on immuno-magnetic isolation and flow cytometry. Journal of Immunological Methods 247, 163-174.
Colombo, M, Raposo, G, and Théry, C (2014). Biogenesis, Secretion, and Intercellular Interactions of Exosomes and Other Extracellular Vesicles. Annu Rev Cell Dev Bi 30, 1-35.
Cordonnier, M, Chanteloup, G, Isambert, N, Seigneuric, R, Fumoleau, P, Garrido, C, and Gobbo, J (2017). Exosomes in cancer theranostic: Diamonds in the rough. Cell Adhesion & Migration 11, 151-163.
Crowell, EF, Gaffuri, A-L, Gayraud-Morel, B, Tajbakhsh, S, and Echard, A (2014). Engulfment of the midbody remnant after cytokinesis in mammalian cells. J Cell Sci 127, 3840-3851.
Crowell, EF, Tinevez, J-Y, and Echard, A (2013). A simple model for the fate of the cytokinesis midbody remnant: implications for remnant degradation by autophagy. Bioessays News Rev Mol Cell Dev Biology 35, 472-481.
Dash, M, Palaniyandi, K, Ramalingam, S, Sahabudeen, S, and Raja, NS (2021). Exosomes isolated from two different cell lines using three different isolation techniques show variation in physical and molecular characteristics. Biochimica et Biophysica Acta (BBA)—Biomembranes 1863, 183490.
D'Avino, PP (2017). Citron kinase—renaissance of a neglected mitotic kinase. J Cell Sci 130, 1701-1708.
D'Avino, PP, and Capalbo, L (2016). Regulation of midbody formation and function by mitotic kinases. Seminars in Cell & Developmental Biology 53, 57-63.
Dellar, ER, Hill, C, Melling, GE, Carter, DRF, and Baena-Lopez, LA (2022). Unpacking extracellular vesicles: RNA cargo loading and function. Journal of Extracellular Biology 1.
Deun, JV, Hendrix, A, and consortium, O behalf of the E-T (2017). Is your article EV-TRACKed? Journal of Extracellular Vesicles 6, 1379835.
Dionne, LK, Wang, X-J, and Prekeris, R (2015). Midbody: from cellular junk to regulator of cell polarity and cell fate. Curr Opin Cell Biol 35, 51-58.
Dongen, HM van, Masoumi, N, Witwer, KW, and Pegtel, DM (2016). Extracellular Vesicles Exploit Viral Entry Routes for Cargo Delivery. Microbiology and Molecular Biology Reviews: MMBR 80, 369-386.
Donovan, TA, Moore, FM, Bertram, CA, Luong, R, Bolfa, P, Klopfleisch, R, Tvedten, H, Salas, EN, Whitley, DB, Aubreville, M, et al. (2021). Mitotic Figures-Normal, Atypical, and Imposters: A Guide to Identification. Veterinary Pathology 58, 243-257.
Douglas, ME, Davies, T, Joseph, N, and Mishima, M (2010). Aurora B and 14-3-3 Coordinately Regulate Clustering of Centralspindlin during Cytokinesis. Current Biology 20, 927-933.
Doyle, LM, and Wang, MZ (2019). Overview of Extracellular Vesicles, Their Origin, Composition, Purpose, and Methods for Exosome Isolation and Analysis. Cells 8, 727.
Echard, A, Hickson, GRX, Foley, E, and O'Farrell, PH (2004). Terminal Cytokinesis Events Uncovered after an RNAi Screen. Current Biology 14, 1685-1693.
Enam, SU, Zinshteyn, B, Goldman, DH, Cassani, M, Livingston, NM, Seydoux, G, and Green, R (2020). Puromycin reactivity does not accurately localize translation at the subcellular level. ELife 9, e60303.
Fang, X, Duan, Y, Adkins, GB, Pan, S, Wang, H, Liu, Y, and Zhong, W (2018). Highly Efficient Exosome Isolation and Protein Analysis by an Integrated Nanomaterial-Based Platform. Analytical Chemistry 90, 2787-2795.
Farmer, T (2022). New signaling kid on the block: the role of the postmitotic midbody in polarity, stemness, and proliferation. Mol Biol Cell 33, pe2.
Farmer, T, Vaeth, KF, Han, K-J, Goering, R, Taliaferro, MJ, and Prekeris, R (2023). The role of midbody-associated mRNAs in regulating abscission. J Cell Biol 222, e202306123.
Gandham, S, Su, X, Wood, J, Nocera, AL, Alli, SC, Milane, L, Zimmerman, A, Amiji, M, and Ivanov, AR (2020). Technologies and Standardization in Research on Extracellular Vesicles. Trends in Biotechnology 38, 1066-1098.
Ghossoub, R, Lembo, F, Rubio, A, Gaillard, CB, Bouchet, J, Vitale, N, Slavík, J, Machala, M, and Zimmermann, P (2014). Syntenin-ALIX exosome biogenesis and budding into multivesicular bodies are controlled by ARF6 and PLD2. Nature Communications 5, 3477.
Guru, KTP, Sreeja, JS, Dharmapal, D, Sengupta, S, and Basu, PK (2022). Novel Gold Nanoparticle-Based Quick Small-Exosome Isolation Technique from Serum Sample at a Low Centrifugal Force. Nanomaterials-Basel 12, 1660.
Gurunathan, S, Kang, M-H, and Kim, J-H (2021). A Comprehensive Review on Factors Influences Biogenesis, Functions, Therapeutic and Clinical Implications of Exosomes. Int J Nanomed 16, 1281-1312.
Gurung, S, Perocheau, D, Touramanidou, L, and Baruteau, J (2021). The exosome journey: from biogenesis to uptake and intracellular signalling. Cell Communication and Signaling 19, 47.
Hanayama, R (2020). Emerging roles of extracellular vesicles in physiology and disease. The Journal of Biochemistry.
Helwa, I, Cai, J, Drewry, MD, Zimmerman, A, Dinkins, MB, Khaled, ML, Seremwe, M, Dismuke, WM, Bieberich, E, Stamer, WD, et al. (2017). A Comparative Study of Serum Exosome Isolation Using Differential Ultracentrifugation and Three Commercial Reagents. PLoS ONE 12, c0170628.
Hemler, ME (2001). Specific tetraspanin functions. The Journal of Cell Biology 155, 1103-1108.
Hessvik, NP, and Llorente, A (2018). Current knowledge on exosome biogenesis and release. Cellular and Molecular Life Sciences 75, 193-208.
Hinger, SA, Cha, DJ, Franklin, JL, Higginbotham, JN, Dou, Y, Ping, J, Shu, L, Prasad, N, Levy, S, Zhang, B, et al. (2018). Diverse Long RNAs Are Differentially Sorted into Extracellular Vesicles Secreted by Colorectal Cancer Cells. Cell Reports 25, 715-725.e4.
Hu, C-K, Coughlin, M, and Mitchison, TJ (2012). Midbody assembly and its regulation during cytokinesis. Molecular Biology of the Cell 23, 1024-1034.
Ibrahim, A, Lashen, A, Toss, M, Mihai, R, and Rakha, E (2022). Assessment of mitotic activity in breast cancer: revisited in the digital pathology era. Journal of Clinical Pathology 75, 365-372.
Ikawa, K, Satou, A, Fukuhara, M, Matsumura, S, Sugiyama, N, Goto, H, Fukuda, M, Inagaki, M, Ishihama, Y, and Toyoshima, F (2014). Inhibition of endocytic vesicle fusion by Plk1-mediated phosphorylation of vimentin during mitosis. Cell Cycle 13, 126-137.
Janas, AM, Sapoń, K, Janas, T, Stowell, MHB, and Janas, T (2016). Exosomes and other extracellular vesicles in neural cells and neurodegenerative diseases. Biochimica et Biophysica Acta (BBA)—Biomembranes 1858, 1139-1151.
Janisch, KM, McNeely, KC, Dardick, JM, Lim, SH, and Dwyer, ND (2017). Kinesin-6 KIF20B is required for efficient cytokinetic furrowing and timely abscission in human cells. Mol Biol Cell 29, 166-179.
Jeppesen, DK, Zhang, Q, Franklin, JL, and Coffey, RJ (2023). Extracellular vesicles and nanoparticles: emerging complexities. Trends in Cell Biology.
Ji, X, Huang, S, Zhang, J, Bruce, TF, Tan, Z, Wang, D, Zhu, J, Marcus, RK, and Lubman, DM (2020). A novel method of high-purity extracellular vesicle enrichment from microliter-scale human serum for proteomic analysis. Electrophoresis.
Jong, OGD, Balkom, BWMV, Schiffelers, RM, Bouten, CVC, and Verhaar, MC (2014). Extracellular Vesicles: Potential Roles in Regenerative Medicine. Frontiers in Immunology 5, 608.
Jung, GI, Londoño-Vásquez, D, Park, S, Skop, AR, Balboula, AZ, and Schindler, K (2023). An oocyte meiotic midbody cap is required for developmental competence in mice. Nat Commun 14, 7419.
Kalluri, R, and LeBleu, VS (2020). The biology, function, and biomedical applications of exosomes. Sci New York N Y 367, eaau6977.
Karbanová, J, Deniz, IA, Wilsch-Bräuninger, M, Couto, RA de S, Fargeas, CA, Santos, MF, Lorico, A, and Corbeil, D (2024). Extracellular lipidosomes containing lipid droplets and mitochondria are released during melanoma cell division. Cell Commun Signal 22, 57.
Kowal, EJK, Ter-Ovanesyan, D, Regev, A, and Church, GM (2017). Extracellular Vesicles, Methods and Protocols. Methods in Molecular Biology 1660, 143-152.
Kowal, J, Arras, G, Colombo, M, Jouve, M, Morath, JP, Primdal-Bengtson, B, Dingli, F, Loew, D, Tkach, M, and Théry, C (2016). Proteomic comparison defines novel markers to characterize heterogeneous populations of extracellular vesicle subtypes. Proc Natl Acad Sci 113, E968-E977.
Kuriyama, R, Gustus, C, Terada, Y, Uetake, Y, and Matuliene, J (2002). CHO1, a mammalian kinesin-like protein, interacts with F-actin and is involved in the terminal phase of cytokinesis. The Journal of Cell Biology 156, 783-790.
Latifkar, A, Hur, YH, Sanchez, JC, Cerione, RA, and Antonyak, MA (2019). New insights into extracellular vesicle biogenesis and function. Journal of Cell Science 132, jcs222406.
Lee, S-S, Won, J-H, Lim, GJ, Han, J, Lee, JY, Cho, K-O, and Bae, Y-K (2019). A novel population of extracellular vesicles smaller than exosomes promotes cell proliferation. Cell Communication and Signaling 17, 95.
Lehmann, BD, Paine, MS, Brooks, AM, McCubrey, JA, Renegar, RH, Wang, R, and Terrian, DM (2008). Senescence-Associated Exosome Release from Human Prostate Cancer Cells. Cancer Res 68, 7864-7871.
Li, Y, Kanao, E, Yamano, T, Ishihama, Y, and Imami, K (2023). TurboID-EV: Proteomic Mapping of Recipient Cellular Proteins Proximal to Small Extracellular Vesicles. Anal Chem 95, 14159-14164.
Li, Y, Zhao, J, Yu, S, Wang, Z, He, X, Su, Y, Guo, T, Sheng, H, Chen, J, Zheng, Q, et al. (2019). Extracellular Vesicles Long RNA Sequencing Reveals Abundant mRNA, circRNA, and IncRNA in Human Blood as Potential Biomarkers for Cancer Diagnosis. Clinical Chemistry 65, 798-808.
Liegertová, M, and Janoušková, O (2023). Bridging the extracellular vesicle knowledge gap: insights from non-mammalian vertebrates, invertebrates, and early-diverging metazoans. Front Cell Dev Biol 11, 1264852.
Lim, J, Choi, M, Lee, H, Kim, Y-H, Han, J-Y, Lee, ES, and Cho, Y (2019). Direct isolation and characterization of circulating exosomes from biological samples using magnetic nanowires. Journal of Nanobiotechnology 17, 1.
Lischnig, A, Bergqvist, M, Ochiya, T, and Lässer, C (2022). Quantitative Proteomics Identifies Proteins Enriched in Large and Small Extracellular Vesicles. Molecular & Cellular Proteomics 21, 100273.
Loomis, RJ, Holmes, DA, Elms, A, Solski, PA, Der, CJ, and Su, L (2006). Citron Kinase, a RhoA Effector, Enhances HIV-1 Virion Production by Modulating Exocytosis. Traffic 7, 1643-1653.
Luo, T, Chen, S, Qiu, Z, Miao, Y, Ding, Y, Pan, X, Li, Y, Lei, Q, and Guo, A (2022). Transcriptomic Features in a Single Extracellular Vesicle via Single-Cell RNA Sequencing. Small Methods 6, e2200881.
Maacha, S, Bhat, AA, Jimenez, L, Raza, A, Haris, M, Uddin, S, and Grivel, J-C (2019). Extracellular vesicles-mediated intercellular communication: roles in the tumor microenvironment and anti-cancer drug resistance. Molecular Cancer 18, 55.
Maas, SLN, Breakefield, XO, and Weaver, AM (2017). Extracellular Vesicles: Unique Intercellular Delivery Vehicles. Trends in Cell Biology 27, 172-188.
Makarova, J, Turchinovich, A, Shkurnikov, M, and Tonevitsky, A (2021). Extracellular miRNAs and Cell-Cell Communication: Problems and Prospects. Trends in Biochemical Sciences.
Makyio, H, Ohgi, M, Takei, T, Takahashi, S, Takatsu, H, Katoh, Y, Hanai, A, Ueda, T, Kanaho, Y, Xie, Y, et al. (2012). Structural basis for Arf6-MKLP1 complex formation on the Flemming body responsible for cytokinesis. EMBO J 31, 2590-2603.
Mardi, N, Haiaty, S, Rahbarghazi, R, Mobarak, H, Milani, M, Zarebkohan, A, and Nouri, M (2023). Exosomal transmission of viruses, a two-edged biological sword. Cell Commun Signal 21, 19.
Margolis, L, and Sadovsky, Y (2019). The biology of extracellular vesicles: The known unknowns. PLOS Biology 17, e3000363.
Markina-Iñarrairaegui, A, Pantazopoulou, A, Espeso, EA, and Peñalva, MA (2013). The Aspergillus nidulans Peripheral ER: Disorganization by ER Stress and Persistence during Mitosis. PLoS ONE 8, e67154.
Martínez-Greene, JA, Hernández-Ortega, K, Quiroz-Baez, R, Resendis-Antonio, O, Pichardo-Casas, I, Sinclair, DA, Budnik, B, Hidalgo-Miranda, A, Uribe-Querol, E, Ramos-Godínez, M del P, et al. (2021). Quantitative proteomic analysis of extracellular vesicle subgroups isolated by an optimized method combining polymer-based precipitation and size exclusion chromatography. J Extracell Vesicles 10, e12087.
Martins, S de T, and Alves, LR (2020). Extracellular Vesicles in Viral Infections: Two Sides of the Same Coin? Frontiers in Cellular and Infection Microbiology 10, 593170.
Martins, TS, Catita, J, Rosa, IM, Silva, OAB da Ce, and Henriques, AG (2018). Exosome isolation from distinct biofluids using precipitation and column-based approaches. PLOS ONE 13, e0198820.
Mathieu, M, Martin-Jaular, L, Lavieu, G, and Théry, C (2019). Specificities of secretion and uptake of exosomes and other extracellular vesicles for cell-to-cell communication. Nat Cell Biol 21, 9-17.
Mathivanan, S, Ji, H, and Simpson, RJ (2010). Exosomes: Extracellular organelles important in intercellular communication. Journal of Proteomics 73, 1907-1920.
Matuliene, J, and Kuriyama, R (2002). Kinesin-like Protein CHO1 Is Required for the Formation of Midbody Matrix and the Completion of Cytokinesis in Mammalian Cells. Molecular Biology of the Cell 13, 1832-1845.
McAndrews, KM, and Kalluri, R (2019). Mechanisms associated with biogenesis of exosomes in cancer. Mol Cancer 18, 52.
Mishima, M, Pavicic, V, Grüneberg, U, Nigg, EA, and Glotzer, M (2004). Cell cycle regulation of central spindle assembly. Nature 430, 908-913.
Mizenko, RR, Brostoff, T, Rojalin, T, Koster, HJ, Swindell, HS, Leiserowitz, GS, Wang, A, and Carney, RP (2021). Tetraspanins are unevenly distributed across single extracellular vesicles and bias sensitivity to multiplexed cancer biomarkers. Journal of Nanobiotechnology 19, 250.
Mullins, JM, and McIntosh, JR (1979). Birefringence of the mammalian midbody. Exp Cell Res 121, 395-399.
Mullins, JM, and McIntosh, JR (1982). Isolation and initial characterization of the mammalian midbody. J Cell Biology 94, 654-661.
Niel, G van, Carter, DRF, Clayton, A, Lambert, DW, Raposo, G, and Vader, P (2022). Challenges and directions in studying cell-cell communication by extracellular vesicles. Nat Rev Mol Cell Biol 23, 369-382.
Niel, G van, D'Angelo, G, and Raposo, G (2018). Shedding light on the cell biology of extracellular vesicles. Nat Rev Mol Cell Bio 19, 213-228.
Nikonorova, IA, Wang, J, Cope, AL, Tilton, PE, Power, KM, Walsh, JD, Akella, JS, Krauchunas, AR, Shah, P, and Barr, MM (2022). Isolation, profiling, and tracking of extracellular vesicle cargo in Caenorhabditis elegans. Curr Biol.
O'Brien, J, Hayder, H, Zayed, Y, and Peng, C (2018). Overview of MicroRNA Biogenesis, Mechanisms of Actions, and Circulation. Frontiers in Endocrinology 9, 402.
O'Brien, K, Breyne, K, Ughetto, S, Laurent, LC, and Breakefield, XO (2020). RNA delivery by extracellular vesicles in mammalian cells and its applications. Nature Reviews Molecular Cell Biology 21, 585-606.
Oksvold, MP, Neurauter, A, and Pedersen, KW (2014). RNA Interference, Challenges and Therapeutic Opportunities. Methods in Molecular Biology 1218, 465-481.
Otegui, MS, Verbrugghe, KJ, and Skop, AR (2005). Midbodies and phragmoplasts: analogous structures involved in cytokinesis. Trends Cell Biol 15, 404-413.
Park, S, Dahn, R, Kurt, E, Presle, A, VanDenHeuvel, K, Moravec, C, Jambhekar, A, Olukoga, O, Shepherd, J, Echard, A, et al. (2023a). The mammalian midbody and midbody remnant are assembly sites for RNA and localized translation. Dev Cell.
Park, S, Patel, SA, Torr, EE, Dureke, A-GN, McIntyre, AM, and Skop, AR (2023b). A protocol for isolating and imaging large extracellular vesicles or midbody remnants from mammalian cell culture. STAR Protoc 4, 102562.
Patel, GK, Khan, MA, Zubair, H, Srivastava, SK, Khushman, M, Singh, S, and Singh, AP (2019). Comparative analysis of exosome isolation methods using culture supernatant for optimum yield, purity and downstream applications. Scientific Reports 9, 5335.
Peterman, E, Gibieža, P, Schafer, J, Skeberdis, VA, Kaupinis, A, Valius, M, Heiligenstein, X, Hurbain, I, Raposo, G, and Prekeris, R (2019). The post-abscission midbody is an intracellular signaling organelle that regulates cell proliferation. Nature Communications 10, 3181.
Peterman, E, and Prekeris, R (2019). The postmitotic midbody: Regulating polarity, stemness, and proliferation. J Cell Biology 218, 3903-3911.
Pinheiro, D, and Bellaïche, Y (2014). Making the Most of the Midbody Remnant: Specification of the Dorsal-Ventral Axis. Dev Cell 28, 219-220.
Pitt, JM, Kroemer, G, and Zitvogel, L (2016). Extracellular vesicles: masters of intercellular communication and potential clinical interventions. Journal of Clinical Investigation 126, 1139-1143.
Pol, E van der, Böing, AN, Harrison, P, Sturk, A, and Nieuwland, R (2012). Classification, Functions, and Clinical Relevance of Extracellular Vesicles. Pharmacological Reviews 64, 676-705.
Qian, F, Huang, Z, Zhong, H, Lei, Q, Ai, Y, Xie, Z, Zhang, T, Jiang, B, Zhu, W, Sheng, Y, et al. (2022). Analysis and Biomedical Applications of Functional Cargo in Extracellular Vesicles. ACS Nano 16, 19980-20001.
Rada, P, Hrdý, I, Zdrha, A, Narayanasamy, RK, Smutná, T, Horáčková, J, Harant, K, Beneš, V, Ong, S-C, Tsai, C-Y, et al. (2022). Double-Stranded RNA Viruses Are Released From Trichomonas vaginalis Inside Small Extracellular Vesicles and Modulate the Exosomal Cargo. Frontiers in Microbiology 13, 893692.
Rai, A, Greening, DW, Xu, R, Chen, M, Suwakulsiri, W, and Simpson, RJ (2021). Secreted midbody remnants are a class of extracellular vesicles molecularly distinct from exosomes and microparticles. Communications Biology 4, 400.
Raposo, G, and Stahl, PD (2019). Extracellular vesicles: a new communication paradigm? Nature Reviews Molecular Cell Biology 20, 509-510.
Rider, MA, Hurwitz, SN, and Meckes, DG (2016). ExtraPEG: A Polyethylene Glycol-Based Method for Enrichment of Extracellular Vesicles. Sci Rep-Uk 6, 23978.
Rodosthenous, RS, Hutchins, E, Reiman, R, Yeri, AS, Srinivasan, S, Whitsett, TG, Ghiran, I, Silverman, MG, Laurent, LC, Keuren-Jensen, KV, et al. (2020). Profiling Extracellular Long RNA Transcriptome in Human Plasma and Extracellular Vesicles for Biomarker Discovery. Iscience 23, 101182.
Sandim, V, and Monteiro, RQ (2020). Extracellular vesicle fingerprinting: the next generation for cancer diagnosis? Signal Transduction and Targeted Therapy 5, 263.
Skop, AR, Liu, H, Yates, J, Meyer, BJ, and Heald, R (2004). Dissection of the Mammalian Midbody Proteome Reveals Conserved Cytokinesis Mechanisms. Science 305, 61-66.
Skottvoll, FS, Berg, HE, Bjrseth, K, Lund, K, Roos, N, Bekhradnia, S, Thiede, B, Sandberg, C, Vik-Mo, EO, Roberg-Larsen, H, et al. (2018). Ultracentrifugation versus kit exosome isolation: nanoLCMS and other tools reveal similar performance biomarkers, but also contaminations. Future Science OA 5, FSO359.
Sung, BH, Parent, CA, and Weaver, AM (2021). Extracellular vesicles: Critical players during cell migration. Developmental Cell 56, 1861-1874.
Suwakulsiri, W, Xu, R, Chen, M, Shafiq, A, Greening, D, and Simpson, R (2023). Comparative proteomic analysis of three major extracellular classes secreted from human adenocarcinoma and metastatic colorectal cancer cells: exosomes, microparticles and shed midbody remnants.
Suwakulsiri, W, Xu, R, Rai, A, Chen, M, Shafiq, A, Greening, DW, and Simpson, RJ (2024). Transcriptomic analysis and fusion gene identifications of midbody remnants released from colorectal cancer cells reveals they are molecularly distinct from exosomes and microparticles. PROTEOMICS, c2300058.
Taheri, B, Zarei-Behjani, Z, Babaei, A, and Moradkhan, FM (2023). Extracellular Vesicles: a Trojan Horse Delivery Method for Systemic Administration of Oncolytic Viruses. Regenerative Engineering and Translational Medicine, 1-11.
Teng, F, and Fussenegger, M (2021). Shedding Light on Extracellular Vesicle Biogenesis and Bioengineering. Adv Sci 8, 2003505.
Termini, CM, and Gillette, JM (2017). Tetraspanins Function as Regulators of Cellular Signaling. Frontiers in Cell and Developmental Biology 5, 34.
Tetta, C, Ghigo, E, Silengo, L, Deregibus, MC, and Camussi, G (2013). Extracellular vesicles as an emerging mechanism of cell-to-cell communication. Endocrine 44, 11-19.
Théry, C, Witwer, KW, Aikawa, E, Alcaraz, MJ, Anderson, JD, Andriantsitohaina, R, Antoniou, A, Arab, T, Archer, F, Atkin-Smith, GK, et al. (2018). Minimal information for studies of extracellular vesicles 2018 (MISEV2018): a position statement of the International Society for Extracellular Vesicles and update of the MISEV2014 guidelines. J Extracell Vesicles 7, 1535750.
Tkach, M, and Théry, C (2016). Communication by Extracellular Vesicles: Where We Are and Where We Need to Go. Cell 164, 1226-1232.
Tosar, JP, Witwer, K, and Cayota, A (2021). Revisiting Extracellular RNA Release, Processing, and Function. Trends in Biochemical Sciences.
Urezkova, MM, Semiglazova, T, Artemyeva, A, and Kudaybergenova, A (2021). Ki-67 and mitotic index in triple-positive breast cancer. J Clin Oncol 39, e12551-e12551.
Valadi, H, Ekström, K, Bossios, A, Sjöstrand, M, Lee, JJ, and Lötvall, JO (2007). Exosome-mediated transfer of mRNAs and microRNAs is a novel mechanism of genetic exchange between cells. Nature Cell Biology 9, 654-659.
Vassilev, LT, Tovar, C, Chen, S, Knezevic, D, Zhao, X, Sun, H, Heimbrook, DC, and Chen, L (2006). Selective small-molecule inhibitor reveals critical mitotic functions of human CDK1. Proc Natl Acad Sci 103, 10660-10665.
Verweij, FJ, Balaj, L, Boulanger, CM, Carter, DRF, Compeer, EB, D'Angelo, G, Andaloussi, SE, Goetz, JG, Gross, JC, Hyenne, V, et al. (2021). The power of imaging to understand extracellular vesicle biology in vivo. Nature Methods, 1-14.
Villa, F, Quarto, R, and Tasso, R (2019). Extracellular Vesicles as Natural, Safe and Efficient Drug Delivery Systems. Pharmaceutics 11, 557.
White, EA, and Glotzer, M (2012). Centralspindlin: at the heart of cytokinesis. Cytoskeleton (Hoboken, NJ) 69, 882-892.
Wu, P, Zhang, B, Ocansey, DKW, Xu, W, and Qian, H (2021). Extracellular vesicles: A bright star of nanomedicine. Biomaterials 269, 120467.
Xie, S, Zhang, Q, and Jiang, L (2022). Current Knowledge on Exosome Biogenesis, Cargo-Sorting Mechanism and Therapeutic Implications. Membranes 12, 498.
Xu, R, Rai, A, Chen, M, Suwakulsiri, W, Greening, DW, and Simpson, RJ (2018). Extracellular vesicles in cancer—implications for future improvements in cancer care. Nature Reviews Clinical Oncology 15, 617-638.
Yáñez-Mó, M, Siljander, PR-M, Andreu, Z, Zavec, AB, Borràs, FE, Buzas, EI, Buzas, K, Casal, E, Cappello, F, Carvalho, J, et al. (2015). Biological properties of extracellular vesicles and their physiological functions. Journal of Extracellular Vesicles 4, 27066.
Yoon, J-S, Kim, K, and Palli, SR (2020). Double-stranded RNA in exosomes: Potential systemic RNA interference pathway in the Colorado potato beetle, Leptinotarsa decemlineata. Journal of Asia-Pacific Entomology 23, 1160-1164.
Yoshioka, Y, Konishi, Y, Kosaka, N, Katsuda, T, Kato, T, and Ochiya, T (2013). Comparative marker analysis of extracellular vesicles in different human cancer types. J Extracell Vesicles 2, 20424.
Zaborowski, MP, Balaj, L, Breakefield, XO, and Lai, CP (2015). Extracellular Vesicles: Composition, Biological Relevance, and Methods of Study. BioScience 65, 783-797.
Zhang, H, Freitas, D, Kim, HS, Fabijanic, K, Li, Z, Chen, H, Mark, MT, Molina, H, Martin, AB, Bojmar, L, et al. (2018). Identification of distinct nanoparticles and subsets of extracellular vesicles by asymmetric flow field-flow fractionation. Nat Cell Biol 20, 332-343.
Zhang, Q, Jeppesen, DK, Higginbotham, JN, Franklin, JL, and Coffey, RJ (2023). Comprehensive isolation of extracellular vesicles and nanoparticles. Nat Protoc, 1-26.
Zhao, F, Cheng, L, Shao, Q, Chen, Z, Lv, X, Li, J, He, L, Sun, Y, Ji, Q, Lu, P, et al. (2020). Characterization of serum small extracellular vesicles and their small RNA contents across humans, rats, and mice. Scientific Reports 10, 4197.
Zhu, C, Bossy-Wetzel, E, and Jiang, W (2005). Recruitment of MKLP1 to the spindle midzone/midbody by INCENP is essential for midbody formation and completion of cytokinesis in human cells. Biochem J 389, 373-381

1. A method of isolating midbodies, midbody remnants or large extracellular vesicles (lEV), the method comprising combining a polyethylene glycol (PEG) solution with a biological sample comprising midbodies or midbody remnants, incubating the PEG solution and the biological sample for at least 4 hours and recovering the midbodies, midbody remnants or lEV, wherein the PEG solution comprises between 0.5 and 5% PEG.

2. The method of claim 1, wherein the biological sample is selected from the group consisting of conditioned media, plasma, serum, cerebral spinal fluid, urine, blood, saliva and tissue.

3. The method of claim 1, wherein the PEG solution has a final concentration of at least 1% and less than 3%.

4. The method of claim 1, wherein the PEG has a molar weight of between 5000 g/mol and 7000 g/mol.

5. The method of claim 1, wherein the midbodies or midbody remnants are recovered by centrifugation at less than 5000×g for 20 min.

6. The method of claim 1, wherein the PEG solution further comprises nanoparticles.

7. The method of claim 6, wherein the nanoparticles are gold nanoparticles or iron oxide nanoparticles.

8. The method of claim 7, wherein the nanoparticles are gold nanoparticles, the gold nanoparticles are in solution with the PEG solution prior to combination with the biological sample, the incubation is for at least 12 hrs and the midbodies or midbody remnants are recovered by centrifugation at 500×g for 10 min.

9. The method of claim 1, wherein the PEG solution and biological sample are incubated together for approximately 12 hrs or more prior to the recovering step.

10. The method of claim 1, wherein the midbodies, midbody remnants or lEV comprise ribosomal subunits and mRNA and are capable of translating the mRNA to generate protein.

11. The method of claim 1, further comprising labeling the recovered midbodies, midbody remnants or lEV with an MKLP1, CD9, MgcRACGAP1, PLK1, AURK, CITK, ANNEXIN 11, TEX14 or ARC affinity reagent and sorting for the labeled recovered midbodies or midbody remnants.

12. The method of claim 1, further comprising detecting the presence of RNA in the midbodies, midbody remnants or lEV.

13. The method of claim 1, further comprising loading the midbodies, midbody remnants or lEV with a therapeutic cargo.

14. A method for inducing the differentiation of cells, the method comprising contacting a pluripotent cell with the midbodies or midbody remnants produced by the method of claim 1, wherein the pluripotent cell differentiates into the same cell type from which the midbodies or midbody remnants were derived.

15. A method detecting cancer in a subject comprising:
obtaining a biological sample from a subject;
isolating the midbodies or midbody remnants from the biological sample using the method of claim 1; and
using the isolated midbodies or midbody remnants to determine if the subject has cancer.

16. A method of delivering a therapeutic cargo to a target cell comprising collecting midbodies or midbody remnants via the method of claim 1 and contacting a target cell with the collected midbodies or midbody remnants.

17. The method of claim 16, wherein the midbodies or midbody remnants are collected from conditioned media collected from cells modified to express a therapeutic cargo.

18. A method of diagnosing a proliferative disease, the method comprising measuring MKLP1 in a biological sample from a subject and comparing the level of MKLP1 in the biological sample to a level of MKLP1 in a control sample, wherein an increase in the level of MKLP1 in the biological sample as compared to that in the control sample is indicative of the proliferative disease in the subject.

19. The method of claim 18, wherein the proliferative disease is selected from the group consisting of cancer, atherosclerosis, rheumatoid arthritis, psoriasis, pulmonary fibrosis, scleroderma, and cirrhosis of the liver.

20. A method of selecting a large extracellular vesicle (lEV) or a midbody or midbody remnant (MBR) from a sample, the method comprising contacting a sample with an anti-MKLP1 antibody, wherein the portion of the sample that binds to MKLP1 contains the lEV or MBR.

21. The method of claim 20, wherein the portion of the sample that does not bind to the anti-MKLP1 antibody contains a small EV.
